(19) United States
(12) Patent Application Publication
(10) Pub. No.: US 2010/0179343 A1
(43) Pub. Date: Jul. 15, 2010

(54) METHOD OF PRODUCING AN OPTICALLY ACTIVE CYANOHYDRIN DERIVATIVE

(75) Inventors: Wee Chuan Yeo, Singapore (SG); L. L. Christina Chai, Singapore (SG); Selvasothi Selvaratnam, Singapore (SG); Takushi Nagata, Singapore (SG); Kasuhiko Yoshinaga, Singapore (SG)

Correspondence Address: SEED INTELLECTUAL PROPERTY LAW GROUP PLLC, 701 FIFTH AVE, SUITE 5400, SEATTLE, WA, 98104, US

(73) Assignees: AGENCY FOR SCIENCE, TECHNOLOGY AND RESEARCH, Singapore (SG); MITSUI CHEMICALS, INC., Tokyo (JP)

(21) Appl. No.: 12/593,873

(22) PCT Filed: Mar. 29, 2007
(86) PCT No.: PCT/SG2007/000084

§ 371 Date: Mar. 25, 2010

(51) Int. Cl. C07C 253/30

(52) U.S. Cl. 558/276

(57) ABSTRACT

The present invention relates to a method of producing an optically active cyanohydrin derivative, which comprises reacting an aldehyde or an asymmetrical ketone with a cyanating agent in the presence of a Lewis base and a titanium compound produced from a partial hydrolysate of titanium tetraalkoxide and an optically active ligand represented by formula (II) or a titanium oxoalkoxide compound represented by formula (I) [TiₓO_{y}](OR¹)_{4x-2y}, and an optically active ligand represented by formula (II), wherein R¹ is an optionally substituted alkyl group or an optionally substituted aryl group; x is an integer of not less than 2; y is an integer of not less than 1; and y/x satisfies 0.1<y/x≦1.5, wherein R², R³ and R⁴ are independently a hydrogen atom, an alkyl group, an alkenyl group, an aryl group, an aromatic heterocyclic group, an acyl group, an alkoxycarbonyl group or an aryloxycarbonyl group, each of which may be optionally substituent, two or more of R², R³ and R⁴ may be linked together to form a ring, and the ring may have a substituent; and A represents a hydrocarbon containing group with three or more carbon atoms having an asymmetric carbon atom or axial asymmetry.

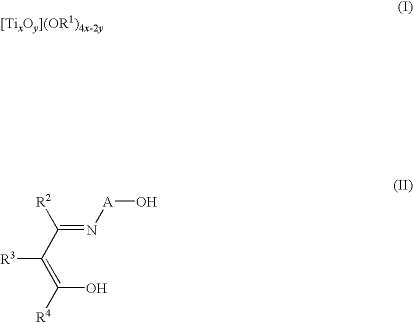

### FIELD OF THE INVENTION

The present invention relates to a method of producing an optically active cyanohydrin derivative.

### BACKGROUND

Optically active cyanohydrins are versatile synthetic precursors in organic synthesis that can be transformed into a variety of compounds and intermediates with commercial and synthetic value. Hence there is a demand for industrially viable asymmetric cyanation catalysts for the synthesis of such compounds. Among the various catalytic systems available, metal catalyzed asymmetric synthesis of cyanohydrins has progressed significantly over the past two decades in terms of synthetic utility, enantioselectivity and general applicability. The majority of such metal catalyzed cyanation systems, however, operate at very low temperatures (such as, for example, −78° C. and −40° C.) and use trimethylsilylcyanide (TMSCN) as the cyanide source, which is relatively expensive, volatile and highly toxic. Most of the catalytic systems also require the use of high catalyst loading and provide a rather low enantiomeric excess (ee) for certain substrates, such as for example aliphatic aldehydes, during the reaction procedure which may not be suitable for large scale production in the kilogram or ton range.

A number of methods for the asymmetric synthesis of cyanohydrins using various catalyst systems have recently been described. See for example, International Patent Application WO2006/041000; Lundgren et al. J. Am. Chem. Soc. 2005, 127, 11592; Wingstrand et al. Pure Appl. Chem 2006, 78, 409; Belokon, et al., Chem. Commun. 2006, 1775; Belokon et al., Org. Lett. 2003, 5, 4505; Belokon et al. Tetrahedron 2004, 60, 10433; Casas et al. Tetrahedron: Asymmetry 2003, 14, 197; Baeza et al. Eur. J. Org. Chem. 2006, 1949; Tian et al., Angew. Chem. Int. Ed. 2002, 41, 3636; Yamagiwa et al. J. Am. Chem. Soc. 2005, 127, 3413, or Gou et al., J. Org. Chem. 2006, 71, 5732.

Hence the availability of catalytic systems which are able to operate at ambient temperature with the use of commercially more viable and less toxic cyanating agents is still desirable. Therefore, there is still a need to provide a method of asymmetric cyanation which can be applied to a wide range of substrates and at the same time achieves a high yield and high enantioselectivity at room temperature, ideally even within a short period of time.

### SUMMARY

In a first aspect, the present invention provides a method of producing an optically active cyanohydrin derivative, which comprises reacting an aldehyde or an unsymmetrical ketone with a cyanating agent in the presence of a Lewis base and a titanium compound.

In a further aspect, the present invention provides a method of producing an optically active cyanohydrin derivative, which comprises reacting an aldehyde or an unsymmetrical ketone with a cyanating agent in the presence of a Lewis base and a titanium compound produced from a partial hydrolysate of titanium tetraalkoxide and an optically active ligand represented by the general formula (II) or a titanium oxoalkoxide compound represented by the general formula (I) and an optically active ligand represented by the general formula (II),

[TiₓO_{y}](OR¹)_{4x-2y} (I)

wherein R¹ is an optionally substituted alkyl group or an optionally substituted aryl group; x is an integer of not less than 2; y is an integer of not less than 1; and y/x satisfies 0.1<y/x≦1.5,

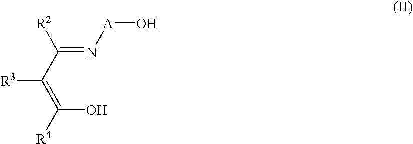

wherein R², R³ and R⁴ are independently a hydrogen atom, an alkyl group, an alkenyl group, an aryl group, an aromatic heterocyclic group, an acyl group, an alkoxycarbonyl group or an aryloxycarbonyl group, each of which may be optionally substituent, two or more of R², R³ and R⁴ may be linked together to form a ring, and the ring may have a substituent; and A represents a hydrocarbon containing group with three or more carbon atoms having at least one asymmetric carbon atom or axial asymmetry.

### BRIEF DESCRIPTION OF THE DRAWINGS

FIG. 1 illustrates the beneficial effect that a partially hydrolyzed titanium compound has over a non-hydrolyzed titanium compound, when used as catalyst, in an illustrative example of a process of the present invention.

### DETAILED DESCRIPTION OF THE INVENTION

In the following description non-limiting embodiments of the process of the invention will be explained.

According to the present invention, it has been surprisingly found that it is possible to conveniently and effectively produce optically active cyanohydrins with a high optical purity with a much less amount of a catalyst and within a much shorter period of time by using the method as recited in independent claim 1 and the claims dependent thereon, as compared to those produced by using the asymmetric catalysis of the past. Such optically active cyanohydrins are typically useful as an intermediate in the synthesis of physiologically active compounds such as medical supplies, agricultural chemicals and the like, functional materials, or synthetic raw materials in fine chemicals and the like.

In the context of the present invention, the term “comprising” or “comprises” means including, but not limited to, whatever follows the word “comprising”. Thus, the use of the term “comprising” indicates that the listed elements are required or mandatory, but that other elements are optional and may or may not be present.

The following terms refer to any groups mentioned in the present invention unless otherwise indicated.

The term “alkyl group” refers to a linear, branched or cyclic alkyl group having 1 to 20 carbon atoms. In one embodiment of the present invention, the alkyl group may have 1 to 15 carbon atoms, for example 1 to 10 carbon atoms. Examples of linear alkyl groups may include, but are not limited to, a methyl group, an ethyl group, a n-propyl group, a n-butyl group, a n-pentyl group, a n-hexyl group, a n-heptyl group, a n-octyl group, a n-nonyl group, a n-decyl group and the like. Examples of branched alkyl groups may include, but are not limited to, an isopropyl group, an isobutyl group, a sec-butyl group, a tert-butyl group, a 2-pentyl group, a 3-pentyl group, an isopentyl group, a neopentyl group, an amyl group and the like. Examples of cyclic alkyl groups may be, but are not limited to, a cyclopropyl group, a cyclobutyl group, a cyclopentyl group, a cyclohexyl group, a cycloheptyl group, a cyclooctyl group and the like.

The term “alkenyl group” refers to a linear, branched or cyclic alkenyl group having 2 to 20 carbon atoms, for example 1 to 10 carbon atoms, wherein at least one carbon-carbon double bond is present. Examples of an alkenyl group may include, but are not limited to, a vinyl group, an allyl group, a crotyl group, a cyclohexenyl group, an isopropenyl group and the like.

The term “alkynyl group” refers to an alkynyl group having 2 to 20 carbon atoms, for example 2 to 10 carbon atoms, wherein at least one carbon-carbon triple bond is present. Examples may include, but are not limited to, an ethynyl group, a 1-propynyl group, a 2-propynyl group, a 1-butynyl group, a 1-pentynyl group and the like.

The term “alkoxy” refers to a linear, branched or cyclic alkoxy group having 1 to 20 carbon atoms, for example 1 to 10 carbon atoms, wherein an alkyl group is bonded to a negatively charged oxygen atom. Examples may include, but are not limited to, a methoxy group, an ethoxy group, a n-propoxy group, an isopropoxy group, a n-butoxy group, a cyclopentyloxy group, a cyclohexyloxy group, a menthyloxy group and the like.

The term “aryl group” refers to an aryl group referring to any functional group or substituent derived from a simple aromatic ring having 6 to 20 carbon atoms. In one embodiment of the present invention, the aryl group may have 6 to 10 carbon atoms. Examples may include, but are not limited to, a phenyl group, a naphthyl group, a biphenyl group, an anthryl group and the like.

The term “aryloxy group” refers to an aryloxy group having 6 to 20 carbon atoms, for example 6 to 10 carbon atoms, wherein an aryl group is bonded to a negatively charged oxygen atom. Examples may include, but are not limited to, a phenoxy group, a naphthyloxy group and the like.

The term “aromatic heterocyclic group” refers to an aromatic heterocyclic group having 3 to 20 carbon atoms, for example 1 to 10 carbon atoms, wherein at least one carbon atom of the aromatic group is replaced by a heteroatom such as nitrogen, oxygen or sulfur. Examples may include, but are not limited to, an imidazolyl group, a furyl group, a thienyl group, a pyridyl group and the like.

The term “non-aromatic heterocyclic group” refers to a non-aromatic heterocyclic group having 4 to 20 carbon atoms, for example 4 to 10 carbon atoms, wherein at least one carbon atom of the non-aromatic group is replaced by a heteroatom such as nitrogen, oxygen or sulfur. Examples may include, but are not limited to, a pyrrolidyl group, a piperidyl group, a tetrahydrofuryl group and the like.

The term “acyl group” refers to an alkylcarbonyl group having 2 to 20 carbon atoms, for example 1 to 10 carbon atoms and an arylcarbonyl group having 6 to 20 carbon atoms, for example 1 to 10 carbon atoms.

The term “alkylcarbonyl group” refers to, but is not limited to, an acetyl group, a propionyl group, a butyryl group, an isobutyryl group, a pivaloyl group and the like.

The term “arylcarbonyl group” refers to, but is not limited to, a benzoyl group, a naphthoyl group, an anthrylcarbonyl group and the like.

The term “alkoxycarbonyl group” refers to a linear, branched or cyclic alkoxycarbonyl group having 2 to 20 carbon atoms, for example 2 to 10 carbon atoms. Examples may include, but are not limited to, a methoxycarbonyl group, an ethoxycarbonyl group, a n-butoxycarbonyl group, a n-octyloxycarbonyl group, an isopropoxycarbonyl group, a tert-butoxycarbonyl group, a cyclopentyloxycarbonyl group, a cyclohexyloxycarbonyl group, a cyclooctyloxycarbonyl group, a L-menthyloxycarbonyl group, a D-menthyloxycarbonyl group and the like.

The term “aryloxycarbonyl group” refers to an aryloxycarbonyl group having 7 to 20 carbon atoms, for example 7 to 15 carbon atoms. Examples may include, but are not limited to, a phenoxycarbonyl group, a α-naphthyloxycarbonyl group and the like.

The term “aminocarbonyl group” refers to an aminocarbonyl group having a hydrogen atom, an alkyl group, an aryl group, and two of the substituents other than a carbonyl group to be bonded to a nitrogen atom may be linked together to form a ring. Examples may include, but are not limited to, an isopropylaminocarbonyl group, a cyclohexylaminocarbonyl group, a tert-butylaminocarbonyl group, a tert-amylaminocarbonyl group, a dimethylaminocarbonyl group, a diethylaminocarbonyl group, a diisopropylaminocarbonyl group, a diisobutylaminocarbonyl group, a dicyclohexylaminocarbonyl group, a tert-butylisopropylaminocarbonyl group, a phenylaminocarbonyl group, a pyrrolidylcarbonyl group, a piperidylcarbonyl group, an indolecarbonyl group and the like.

The term “amino group” refers to organic compounds and a type of functional group that contain nitrogen as the key atom. The term refers to an amino group having a hydrogen atom, a linear, branched or cyclic alkyl group, or an amino group having an aryl group. Two substituents to be bonded to a nitrogen atom may be linked together to form a ring. Examples of the amino group having an alkyl group or an aryl group may include, but are not limited to, an isopropylamino group, a cyclohexylamino group, a tert-butylamino group, a tert-amylamino group, a dimethylamino group, a diethylamino group, a diisopropylamino group, a diisobutylamino group, a dicyclohexylamino group, a tert-butylisopropylamino group, a pyrrolidyl group, a piperidyl group, an indole group and the like.

The term “silyl group” refers to a silyl group having 2 to 20 carbon atoms, wherein the silyl group can be considered as silicon analogue of an alkyl. Examples may include, but are not limited to, a trimethylsilyl group, tert-butyldimethylsilyl group and the like.

The term “siloxy group” refers to a siloxy group having 2 to 20 carbon atoms. Examples may include, but are not limited to, a trimethylsiloxy group, a tert-butyldimethylsiloxy group, a tert-butyldiphenylsiloxy group and the like.

All of the above mentioned groups may be optionally substituted. “Optionally substituted” in the context of the present invention means that at least one hydrogen atom of the above compounds may be replaced by F, Cl, Br, OH, CN, NO₂, NH₂, SO₂, an alkyl group, an aryl group, an aromatic heterocyclic group, a non-aromatic heterocyclic group, an oxygen containing group, a nitrogen containing group, a silicon containing group or the like.

Examples of the oxygen containing group may include, but are not limited to, those having 1 to 20 carbon atoms such as an alkoxy group, an aryloxy group, an alkoxycarbonyl group, an aryloxycarbonyl group, an acyloxy group and the like. Examples of the nitrogen containing group may include, but are not limited to, an amino group having 1 to 20 carbon atoms, an amide group having 1 to 20 carbon atoms, a nitro group, a cyano group and the like. Examples of the silicon containing group may include, but are not limited to, those having 1 to 20 carbon atoms such as a silyl group, a silyloxy group and the like.

Examples of substituted alkyl groups may include, but are not limited to, a chloromethyl group, a 2-chloroethyl group, a trifluoromethyl group, a 2,2,2-trifluoroethyl group, a perfluoroethyl group, a perfluorohexyl, a substituted or unsubstituted aralkyl group such as a benzyl group, a 4-methoxybenzyl group, a 2-phenylethyl group, a cumyl group, a α-naphthylmethyl, a 2-pyridylmethyl group, a 2-furfuryl group, a 3-furfuryl group, a 2-thienylmethyl group, a 2-tetrahydrofurfuryl group, a 3-tetrahydrofurfuryl group, a methoxyethyl group, a phenoxyethyl group, a methoxymethyl group, an isopropoxymethyl group, a tert-butoxymethyl group, a cyclohexyloxymethyl group, a L-menthyloxymethyl group, a D-menthyloxymethyl group, a phenoxymethyl group, a benzyloxymethyl group, a phenoxyethyl group, an acetyloxymethyl group, a 2,4,6-trimethylbenzoyloxymethyl, a 2-(dimethylamino)ethyl group, a 3-(diphenylamino)propyl group, a 2-(trimethylsiloxy)ethyl group and the like.

Examples of substituted alkenyl groups may include, but are not limited to, a 2-chlorovinyl group, a 2,2-dichlorovinyl group, a 3-chloroisopropenyl group or the like.

Examples of substituted alkynyl groups may include, but are not limited to, a 3-chloro-1-propynyl group, a 2-phenylethynyl group, a 3-phenyl-2-propynyl group, a 2-(2-pyridylethynyl) group, a 2-tetrahydrofurylethynyl group, a 2-methoxyethynyl group, a 2-phenoxyethynyl group, a 2-(dimethylamino)ethynyl group, a 3-(diphenylamino)propynyl group, a 2-(trimethylsiloxy)ethynyl group and the like.

Examples of substituted alkoxy groups may include, but are not limited to, a 2,2,2-trifluoroethoxy group, a benzyloxy group, a 4-methoxybenzyloxy group, a 2-phenylethoxy group, a 2-pyridylmethoxy group, a furfuryloxy group, a 2-thienylmethoxy group, a tetrahydrofurfuryloxy group and the like.

Examples of substituted aryl groups may include, but are not limited to, a 4-fluorophenyl group, a pentafluorophenyl group, a 3,5-dimethylphenyl group, a 2,4,6-trimethylphenyl group, a 4-isopropylphenyl group, a 3,5-diisopropylphenyl group, a 2,6-diisopropylphenyl group, a 4-tert-butylphenyl group, a 2,6-di-tent-butylphenyl group, a 4-methoxyphenyl group, a 3,5-dimethoxyphenyl group, a 3,5-diisopropoxyphenyl group, a 2,4,6-triisopropoxyphenyl group, a 2,6-diphenoxyphenyl group, a 4-(dimethylamino)phenyl group, a 4-nitrophenyl group, 3,5-bis(trimethylsilyl)phenyl group, a 3,5-bis(trimethylsiloxy)phenyl group and the like.

Examples of substituted aryloxy groups may include, but are not limited to, a pentafluorophenoxy group, a 2,6-dimethylphenoxy group, a 2,4,6-trimethylphenoxy group, a 2,6-dimethoxyphenoxy group, a 2,6-diisopropoxyphenoxy group, a 4-(dimethylamino)phenoxy group, a 4-cyanophenoxy group, a 2,6-bis(trimethylsilyl)phenoxy group, a 2,6-bis(trimethylsiloxy)phenoxy group and the like.

Examples of substituted aromatic heterocyclic groups may include, but are not limited to, an N-methylimidazolyl group, a 4,5-dimethyl-2-furyl group, a 5-butoxycarbonyl-2-furyl group, a 5-butylaminocarbonyl-2-furyl group, and the like.

Examples of substituted non-aromatic heterocyclic groups may include, but are not limited to, 3-methyl-2-tetrahydrofuranyl group, a N-phenyl-4-piperidyl group, a 3-methoxy-2-pyrrolidyl group and the like.

Examples of substituted alkylcarbonyl group may include, but are not limited to, a trifluoroacetyl group and the like.

Examples of substituted arylcarbonyl groups may include, but are not limited to, a 3,5-dimethylbenzoyl group, a 2,4,6-trimethylbenzoyl group, a 2,6-dimethoxybenzoyl group, a 2,6-diisopropoxybenzoyl group, a 4-(dimethylamino)benzoyl group, a 4-cyanobenzoyl group, a 2,6-bis(trimethylsilyl)benzoyl group, a 2,6-bis(trimethylsiloxy)benzoyl group and the like.

Examples of the alkoxycarbonyl group having a halogen atom include a 2,2,2-trifluoroethoxycarbonyl group, a benzyloxycarbonyl group, a 4-methoxybenzyloxycarbonyl group, a 2-phenylethoxycarbonyl group, a cumyloxycarbonyl group, an α-naphthylmethoxycarbonyl group, a 2-pyridylmethoxycarbonyl group, a furfuryloxycarbonyl group, a 2-thienylmethoxycarbonyl group, a tetrahydrofurfuryloxycarbonyl group, a benzyloxycarbonyl group, a 4-methoxybenzyloxycarbonyl group, a 2-phenylethoxycarbonyl group, a cumyloxycarbonyl group, an α-naphthylmethoxycarbonyl group, a 2-pyridylmethoxycarbonyl group, a furfuryloxycarbonyl group, a 2-thienylmethoxycarbonyl group, a tetrahydrofurfuryloxycarbonyl group and the like.

Examples of substituted aryloxycarbonyl groups may include, but are not limited to, a pentafluorophenoxycarbonyl group, a 2,6-dimethylphenoxycarbonyl group, a 2,4,6-trimethylphenoxycarbonyl group, a 2,6-dimethoxyphenoxycarbonyl group, a 2,6-diisopropoxyphenoxycarbonyl group, a 4-(dimethylamino)phenoxycarbonyl group, a 4-cyanophenoxycarbonyl group, a 2,6-bis(trimethylsilyl)phenoxycarbonyl group, a 2,6-bis(trimethylsiloxy)phenoxycarbonyl group and the like.

Examples of substituted aminocarbonyl groups may include, but are not limited to, a 2-chloroethylaminocarbonyl group, a perfluoroethylaminocarbonyl group, a 4-chlorophenylaminocarbonyl group, a pentafluorophenylaminocarbonyl group, a benzylaminocarbonyl group, a 2-phenylethylaminocarbonyl group, an α-naphthylmethylaminocarbonyl and a 2,4,6-trimethylphenylaminocarbonyl group and the like.

Examples of substituted amino groups may include, but are not limited to, a 2,2,2-trichloroethylamino group, a perfluoroethylamino group, a pentafluorophenylamino group, a benzylamino group, a 2-phenylethylamino group, a α-naphthylmethylamino group, a 2,4,6-trimethylphenylamino group and the like.

In more detail, the present invention provides a method of producing an optically active cyanohydrin derivative, which comprises reacting an aldehyde or an unsymmetrical ketone with a cyanating agent in the presence of a Lewis base and a titanium compound produced from a partial hydrolysate of titanium tetraalkoxide and an optically active ligand represented by the general formula (II) or a titanium oxoalkoxide compound represented by the general formula (I) and an optically active ligand represented by the general formula (II),

[TiₓO_{y}](OR¹)_{4x-2y} (I)

wherein R¹ is an optionally substituted alkyl group or an optionally substituted aryl group; x is an integer of not less than 2; y is an integer of not less than 1; and y/x satisfies 0.1<y/x≦1.5,

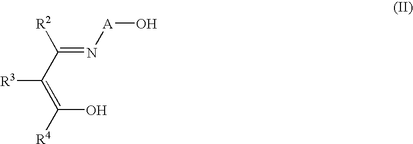

wherein R², R³ and R⁴ are independently a hydrogen atom, an alkyl group, an alkenyl group, an aryl group, an aromatic heterocyclic group, an acyl group, an alkoxycarbonyl group or an aryloxycarbonyl group, each of which may be optionally substituent, two or more of R², R³ and R⁴ may be linked together to form a ring, and the ring may have a substituent; and A represents a hydrocarbon containing group with three or more carbon atoms having an asymmetric carbon atom or axial asymmetry.

The titanium tetraalkoxide compound of the present invention is not particularly limited. In one embodiment of the present invention, the titanium tetraalkoxide compound is represented by the general formula (IV)

[Ti(OR^{a})₄] (IV)

wherein R^{a} is an optionally substituted alkyl group or an optionally substituted aryl group as defined above. In one embodiment of the present invention, R^{a} may be a linear alkyl group as defined above.

In a further embodiment of the present invention, the titanium tetraalkoxide compound may be Ti(OMe)₄, Ti(OEt)₄, Ti(OPrⁿ)₄ or Ti(OBuⁿ)₄.

Further, a titanium oxoalkoxide compound represented by the general formula (I) can also be used for the titanium compound of the present invention

[TiₓO_{y}](OR¹)_{4x-2y} (I).

In the general formula (I), R¹ represents an optionally substituted alkyl group or an optionally substituted aryl group, as already defined above. x is an integer of not less than 2, y is an integer of not less than 1, and y/x satisfies 0.1<y/x≦1.5. It may also be possible to use mixtures of titanium oxoalkoxide compounds, i.e. mixtures of species having a certain range of x and y.

It is known, that by reacting the titanium tetraalkoxide compound represented by the above general formula (IV) with water, titanium tetraalkoxide is partially hydrolyzed to give a titanium oxoalkoxide compound represented by the above general formula (I) (cf. for example, V. W. Day et al. Inorg. Chim. Acta, Vol. 229, p. 391 (1995)). Depending on the kind of alkoxide and the amount of water used for the hydrolysis, the values of x and y in the above general formula (I) are varied, but are not necessarily determined only one-sidedly. So, it is considered that various kinds of titanium oxoalkoxide mixtures are obtained. Further, there has been reported that various titanium oxoalkoxide mixtures can be stably isolated to respective substances in some cases (for example, V. W. Day et al, J. Am. Chem. Soc., Vol. 113, p. 8190 (1991)).

As a raw material of the titanium compound used in the method of the present invention, a reaction mixture of a titanium tetraalkoxide compound with water may be used as it is. Alternatively, a titanium tetraalkoxide compound may be used after it has been isolated from this reaction mixture. In this titanium oxoalkoxide compound, x may be from 2 to 20, for example from 2 to 10. Examples thereof may include, but are not limited to, a titanium alkoxide dimer such as [Ti₂O](OEt)₆, [Ti₂O](O-n-Pr)₆, [Ti₂O](O-n-Bu)₆ and the like; a titanium alkoxide heptamer such as [Ti₇O₄](OEt)₂₀, [Ti₇O₄](O-n-Pr)₂₀, [Ti₇O₄](O-n-Bu)₂₀ and the like; a titanium alkoxide octamer such as [Ti₈O₆](OCH₂Ph)₂₀ and the like; a titanium alkoxide decamer such as [Ti₁₀O₈](OEt)₂₄ and the like; a titanium alkoxide undecamer such as [Ti₁₁O₁₃](O-i-Pr)₁₈ and the like; a titanium alkoxide dodecamer such as [Ti₁₂O₁₆](O-i-Pr)₁₆ and the like; a titanium alkoxide hexadecamer such as [Ti₁₆O₁₆](OEt)₃₂ and the like; and a titanium alkoxide heptadecamer such as [Ti₁₇O₂₄](O-i-Pr)₂₀ and the like.

The titanium compound used in the method of the present invention is produced from a reaction mixture of a partial hydrolysate of titanium tetraalkoxide and an optically active ligand represented by the general formula (II) or a titanium oxoalkoxide compound represented by the above general formula (I) and an optically active ligand represented by the general formula (II),

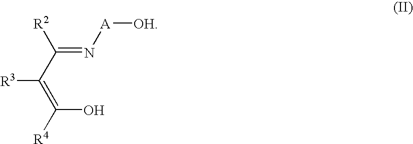

In the above general formula (II), R², R³ and R⁴ are independently a hydrogen atom, an alkyl group, an alkenyl group, an aryl group, an aromatic heterocyclic group, a non-aromatic heterocyclic group, an acyl group, an alkoxycarbonyl group or an aryloxycarbonyl group, each of which may be optionally substituted, wherein alkyl and aryl are as defined above; two or more of R², R³ and R⁴ may be linked together to form a ring, and the ring may have a substituent; and A represents a hydrocarbon containing group with three or more carbon atoms having an asymmetric carbon atom or axial asymmetry.

In one embodiment of the present invention, R², R³ and R⁴ may be an optionally substituted linear, branched or cyclic alkyl group having 1 to 20 carbon atoms, for example 1 to 10 carbon atoms, as defined above. In one embodiment of the present invention, the aryl group in R², R³ and R⁴ may have 6 to 20 carbon atoms, such as 6 to 10 carbon atoms. In one embodiment R² is hydrogen.

Furthermore, two or more of R², R³ and R⁴ may be linked together to form a ring. The ring may be an aliphatic or aromatic hydrocarbon ring. The formed ring may be a condensed ring. In one embodiment of the present invention the aliphatic hydrocarbon ring may be a 10 or less membered ring, such as a 3- to 7-membered ring, for example a 5- or 6-membered ring. The aliphatic hydrocarbon ring may have unsaturated bonds. The aromatic hydrocarbon ring may be, for example, a 6-membered ring, that is, a benzene ring. For example, when R³ and R⁴ are linked together to form —(CH₂)₄— or —CH═CH—CH═CH—, a cyclohexene ring (included in the aliphatic hydrocarbon ring) or a benzene ring (included in the aromatic hydrocarbon ring) is formed, respectively. In one embodiment of the present invention, R³ and R⁴ form a benzene ring.

The ring formed may be optionally substituted as described above. In one embodiment of the present invention, the ring may have one, two or three substituents.

In the above general formula (II), A represents an optically active hydrocarbon containing group with three or more carbon atoms having an asymmetric carbon atom or axial asymmetry. In one embodiment, the optically active hydrocarbon containing group may have 3 to 20 carbon atoms, for example 3 to 10 carbon atoms, which may be optionally substituted.

As the optically active hydrocarbon containing group A as defined above, optically active hydrocarbon containing groups represented by the following general formulae (A-1) to (A-3) are suitable. In the formulae, parts indicated as (N) and (OH) do not belong to A, and represent a nitrogen atom and a hydroxyl group corresponding to those in the above general formula (II) to which A is bonded.

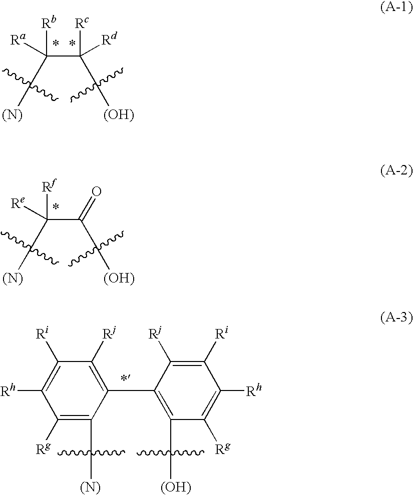

In the above general formula (A-1), R^{a}, R^{b}, R^{c} and R^{d} are each independently a hydrogen atom, an alkyl group, an aryl group, an alkoxycarbonyl group, an aryloxycarbonyl group or an aminocarbonyl group, each of which may be optionally substituted, two or more of R^{a}, R^{b}, R^{c} and R^{d} may be linked together to form a ring and the ring may be optionally substituted; at least one of R^{a}, R^{b}, R^{c} and R^{d} is not hydrogen; both or at least one of the carbon atoms indicated as * become an asymmetric center.

An alkyl group, an aryl group, an alkoxycarbonyl group and an aryloxycarbonyl group in R^{a}, R^{b}, R^{c} and R^{d} are the same as the alkyl group, the aryl group, the alkoxycarbonyl group and the aryloxycarbonyl group as in the above R² to R⁴. In one embodiment of the present invention one of R^{a} or R^{b} and one of R^{c} or R^{d} are hydrogen.

In one embodiment, two or more of R^{a}, R^{b}, R^{c} and R^{d} may be linked together to form a ring. The ring may be an aliphatic hydrocarbon and the formed ring may be further condensed to form a ring. In one embodiment, the ring may be a 3- to 7-membered ring or a 5- or 6-membered ring. For example, when R^{a} and R^{c} are linked together to form —(CH₂)₃—, a 5-membered ring is formed. The thus formed ring may be optionally substituted.

In one embodiment of the present invention, the optically active hydrocarbon containing group represented by the above general formula (A-1) may include those represented by the following formulas (A-1a) to (A-1x), their enantiomers and the like.

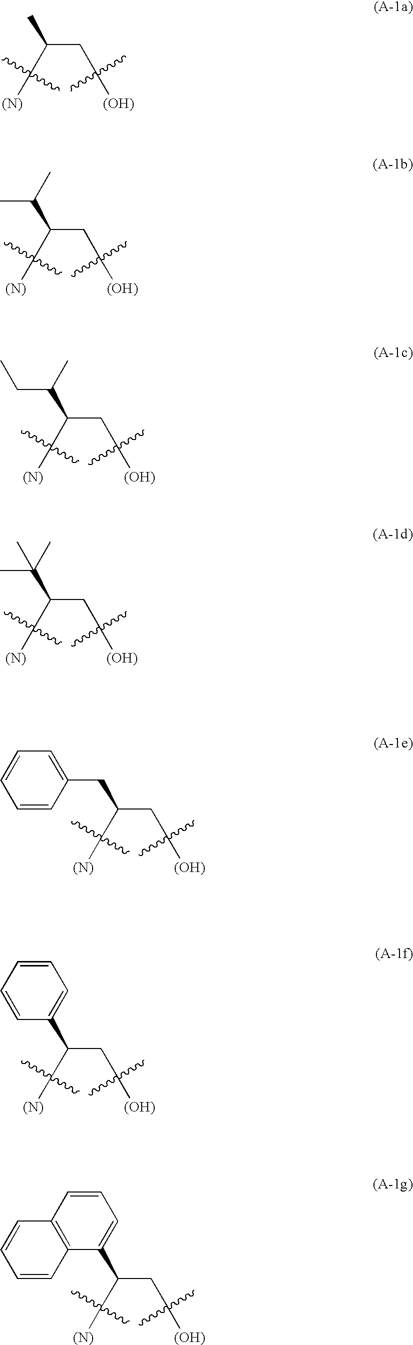

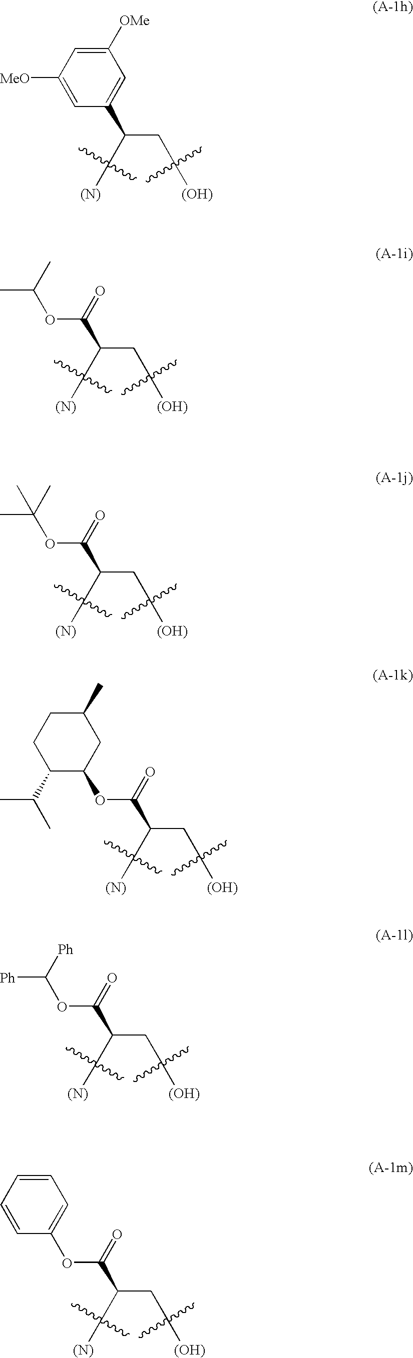

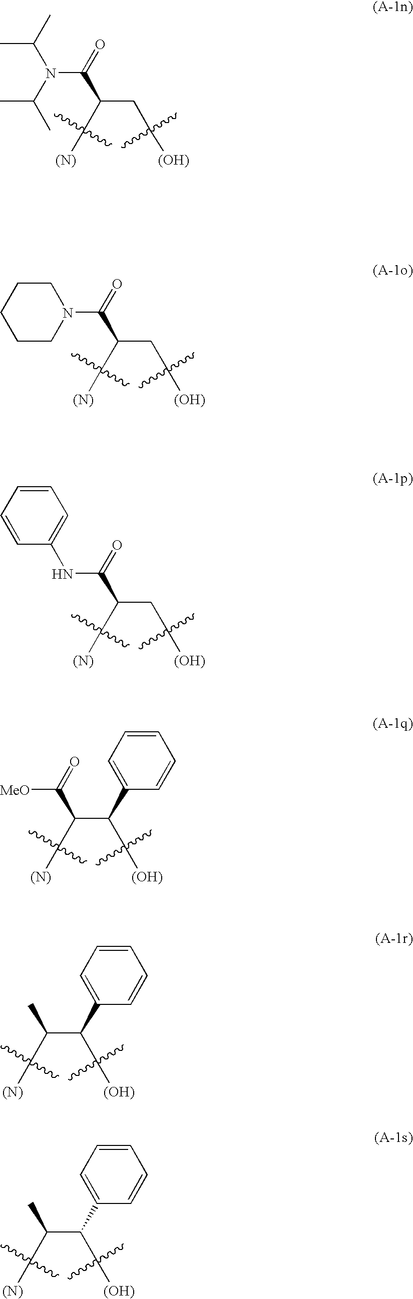

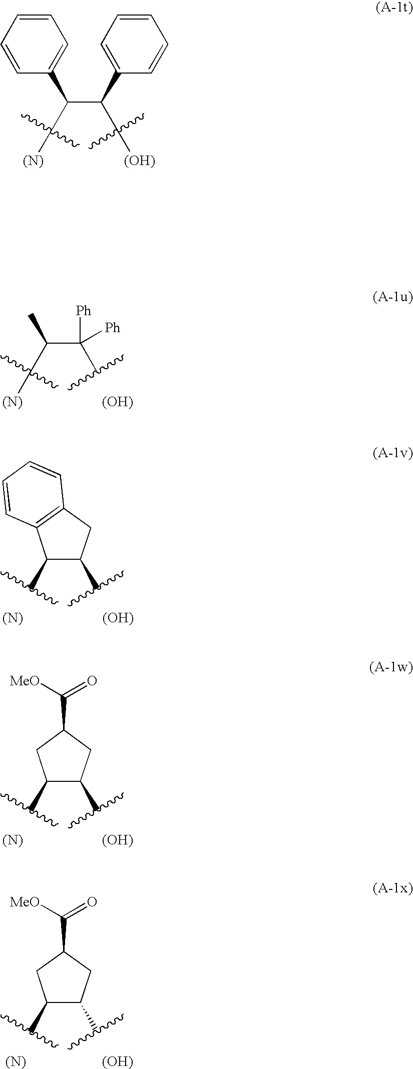

In the above general formula (A-2), R^{e} and R^{f} are independently each a hydrogen atom, an alkyl group or an aryl group, each of which may be optionally substituted. Furthermore, R^{e} and R^{f} are different substituents, and * represents an asymmetric carbon atom. The alkyl group and aryl group in R^{e} and R^{f} are the same as the alkyl group and aryl group in the above R^{a} to R^{d}.

Examples of the optically active hydrocarbon containing group represented by the above general formula (A-2) may include those represented by the following formulas (A-2a) to (A-2p), their enantiomers and the like.

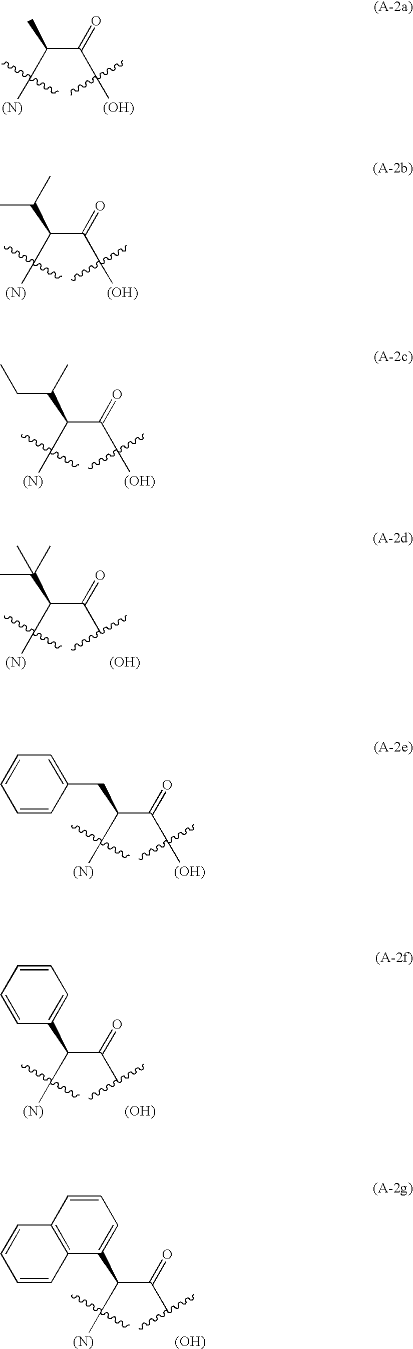

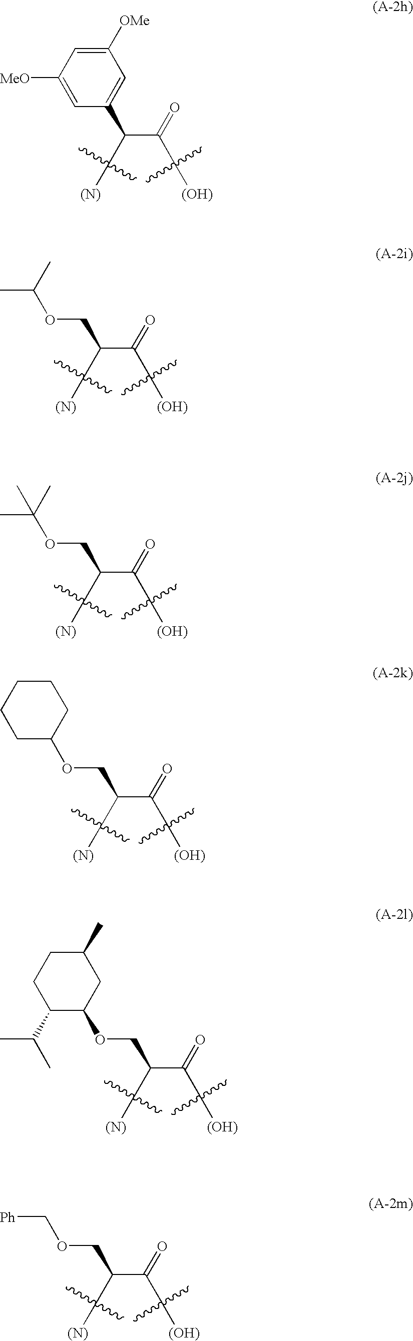

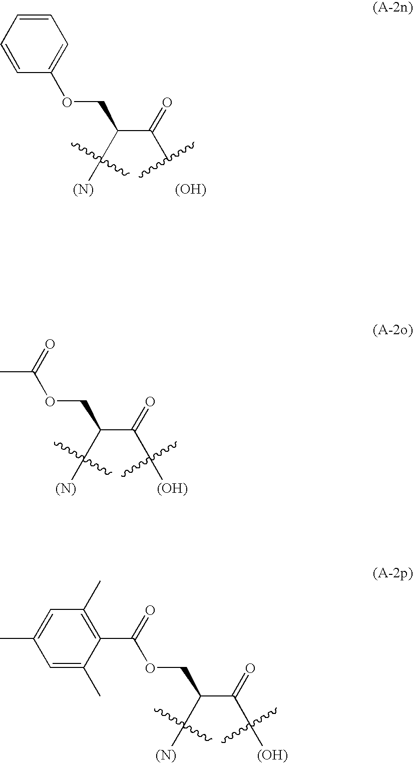

In the above general formula (A-3), R^{g}, R^{h}, Rⁱ and R^{j} may independently be a hydrogen atom, a halogen atom, an alkyl group, an aryl group or an alkoxy group, each of which may be optionally substituted. Further, Rⁱ and R^{j} on the same benzene ring may be linked together or condensed to form a ring and *′ represents an axial asymmetry.

The alkyl group and aryl group in R^{g}, R^{h}, Rⁱ and R^{j} are the same as the alkyl group and the aryl group in the above R² to R⁴. Furthermore, when Rⁱ and R^{j} on the same benzene ring are linked together to form a ring, the ring may be an aliphatic or aromatic hydrocarbon ring, or a non-aromatic heterocyclic containing an oxygen atom. The formed ring may be a condensed ring. In one embodiment, the aliphatic hydrocarbon ring may be a 5- or 6-membered ring, for example a 6-membered ring, that is, a benzene ring. In a further embodiment, the benzene rings may be condensed to form a condensed polycyclic ring such as a naphthalene ring and the like. For example, when Rⁱ and R^{j} are linked together to form —CH═CH—CH═CH—, —(CH₂)₄— or —OCH₂O—, a naphthalene ring, a tetrahydronaphthalene ring or a benzodioxorane ring is formed, respectively. The thus formed ring such as a naphthalene ring, a tetrahydronaphthalene ring, a benzodioxorane ring or the like may be optionally substituted as defined above, for example may have one substituent or two substituents or more substituents.

Examples of the optically active hydrocarbon containing group represented by the above general formula (A-3) may include, but are not limited to, those represented by the following formulas (A-3a) to (A-3c), their enantiomers and the like.

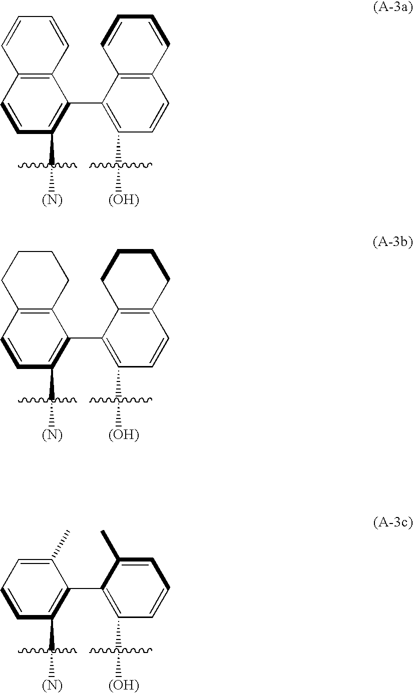

In one embodiment of the present invention the optically active ligand represented by the general formula (II) includes optically active ligands represented by the above general formula (III).

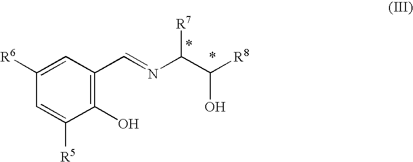

R⁵, R⁶, R⁷ and R⁸ may independently be a hydrogen atom, a halogen atom, an alkyl group, an alkenyl group, an alkynyl group, an aryl group, an aromatic heterocyclic group, a non-aromatic heterocyclic group, an alkoxycarbonyl group, an aryloxycarbonyl group or an aminocarbonyl group, each of which may be optionally substituted, cyano, nitro, OH, an alkoxy group, an amino group, a silyl group or a siloxy group, wherein at least one of R⁷ and R⁸ is not hydrogen, and wherein R⁷ and R⁸ together may form an optically substituted ring having 4 to 8 carbon atoms. Both or at least one of the carbon atoms indicated as * become an asymmetric center. For example, one of R⁷ and R⁸ is hydrogen, whereas the other is an alkyl group.

Furthermore, R⁷ and R⁸ may be linked together to form a ring. In one embodiment, the ring may be an aliphatic or aromatic hydrocarbon ring. The formed ring may be a condensed ring. The aliphatic hydrocarbon ring may be a 10 or less-membered ring, for example a 3- to 7-membered ring, such as a 5- or 6-membered ring. The aliphatic hydrocarbon ring may have unsaturated bonds. The aromatic hydrocarbon ring may be a 6-membered ring, that is, a benzene ring. For example, when R⁷ and R⁸ are linked together to form —(CH₂)₄— or —CH═CH—CH═CH—, a cyclohexene ring (included in the aliphatic hydrocarbon ring) or a benzene ring (included in the aromatic hydrocarbon ring) is formed, respectively. The thus formed ring may be optionally substituted, for example by one group or two or more groups selected from a halogen atom, an alkyl group, an aryl group, an alkoxy group, an aryloxy group, an amino group, a nitro group, a cyano group, a silyl group and a silyloxy group.

In one embodiment of the present invention, the optically active ligand may be

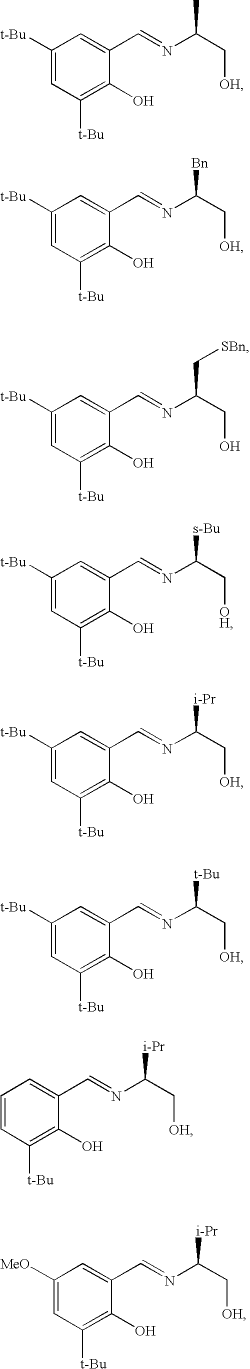

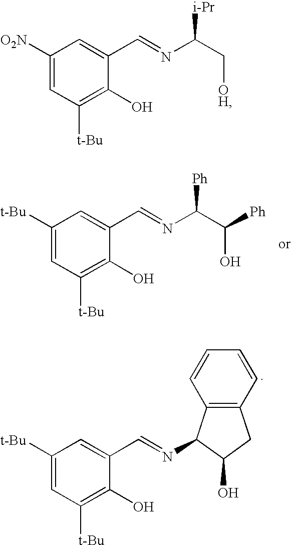

In one embodiment of the present invention, the optically active ligand may be

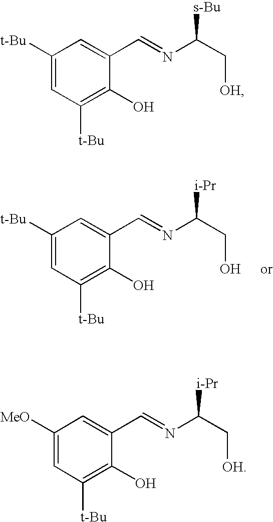

In a still further embodiment, the optically active ligand is

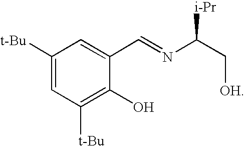

In a further embodiment of the present invention, the optically active ligand according to formula (III) may be a reduced Schiff base ligand.

Examples of such ligands may include, but are not limited to,

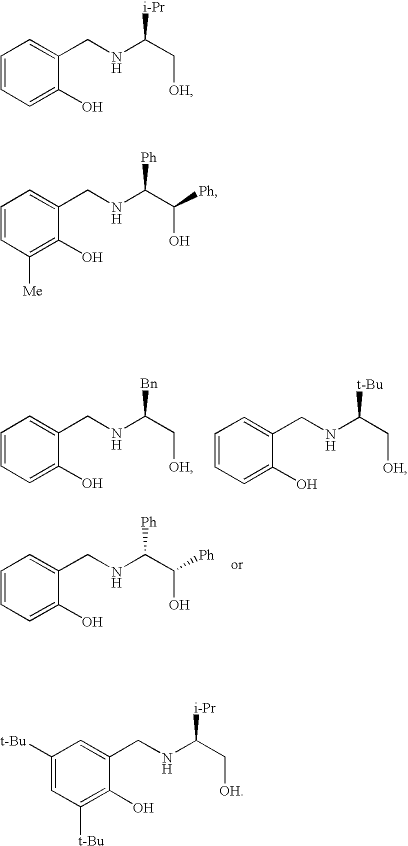

The aforementioned titanium tetraalkoxide compound can be produced according to known methods. For example, it can be produced, in the presence or absence of a base, by adding the corresponding alcohol to titanium tetrachloride in a prescribed amount, stirring the resulting mixture, and then purifying it by distillation. It is also possible to use a solution prepared from titanium tetrachloride and alcohol as it is without purification for the production of an optically active titanium compound.

The titanium oxoalkoxide compound represented by the above general formula (I) can be produced according to known methods. For example, there have been known a method comprising hydrolyzing titanium tetraalkoxide in alcohol (Day, V. W. et al.; J. Am. Chem. Soc., Vol. 113, p. 8190 (1991)), a method comprising reacting titanium tetraalkoxide with a carboxylic acid (Steunou, N. et al; J. Chem. Soc. Dalton Trans., p. 3653 (1999)) and the like. The obtained titanium oxoalkoxide compound may be used as it is without purification for the production of an optically active titanium compound or may be purified according to a known purification method such as recrystallization or the like, prior to use.

The optically active ligand represented by the above general formula (II) can be synthesized, for example, from an optically active amino alcohol and an o-hydroxybenzaldehyde derivative, or from amino alcohol and an o-hydroxyphenyl ketone derivative in one step by a dehydration reaction. The optically active amino alcohol may be obtained, for example, by reducing a carboxylic group of a natural or non-natural α-amino acid and various kinds thereof.

The titanium compound can be produced by reacting the above titanium tetraalkoxide compound represented by the general formula (IV) with water in an organic solvent, and then mixing with the optically active ligand represented by the above general formula (II). The mole ratio of the titanium tetraalkoxide compound, water and the optically active ligand represented by the above general formula (II) may be in the range from 1:0.1:0.1 to 1:2.0:3.0. Any molar ratio within the aforementioned numerical range may be suitable in the present invention.

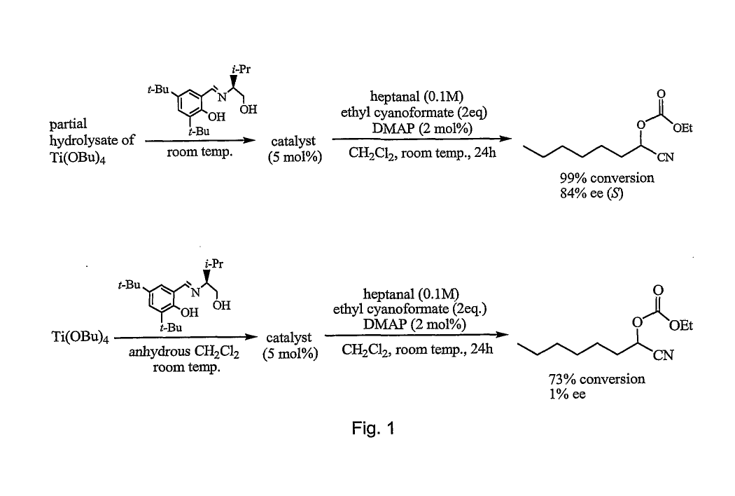

Firstly, a titanium tetraalkoxide compound is reacted with a water source in an organic solvent. The water source (herein referred as “water”) may be, but is not limited to, H₂O, an inorganic salt containing water of crystallization, for example, hydrates such as Na₂B₄O₇.10H₂O, Na₂SO₄.10H₂O, Na₃PO₄.12H₂O, MgSO₄.7H₂O, CuSO₄.5H₂O, FeSO₄.7H₂O, AINa(SO₄)₂.12H₂O or AIK(SO₄)₂.12H₂O and the like. When a moisture-absorbed molecular sieve is used, commercial products such as molecular sieves 3A, 4A and the like exposed to outdoor air may be used, and any of a powder molecular sieve and a pellet molecular sieve can be used. In addition undehydrated silica gel or zeolite may also be used as water source. Further, when an inorganic salt containing water of crystallization or a molecular sieve is used, it can be easily removed by filtering before it is reacted with a ligand. At that time, water may be contained in an amount of from about 0.1 to about 2.0 moles or from about 0.5 to about 1.25 moles, or even about 1 mole, based on 1 mole of the titanium tetraalkoxide compound. In one embodiment, less than 2 mol of water are used for 1 mol of the titanium compound. Water in that amount is added and stirred. At that time, the titanium tetraalkoxide compound may be dissolved in a solvent in advance and water may be diluted in a solvent, prior to addition. Water can also be directly added by a method comprising adding water in mist form, a method comprising using a reaction vessel equipped with a stirrer with high efficiency or the like. Examples of the organic solvent in use may include, but is not limited to, halogenated hydrocarbon solvents such as dichloromethane, chloroform, fluorobenzene, trifluoromethylbenzene and the like; aromatic hydrocarbon solvents such as toluene, xylene and the like; ester solvents such as ethyl acetate and the like; and ether solvents such as tetrahydrofuran, dioxane, diethyl ether, dimethoxyethane and the like. In one embodiment, halogenated solvents or aromatic hydrocarbon solvents may be used. The total amount of the solvent used when water is added may be from about 1 to about 500 ml or from about 10 to about 50 ml, based on 1 millimole of the titanium tetraalkoxide compound. It should be noted that use of the partially hydrolyzed titanium precursor leads to an overall increased conversion rate and enantioselectivity in the further reaction process, as can be taken from FIG. 1.

The temperature when the titanium tetraalkoxide compound is reacted with water is preferably a temperature which does not freeze the solvent. The reaction can usually be carried out at about room temperature, for example, from about 15 to about 30° C. The reaction may however also be carried out by heating depending on the boiling point of the solvent in use. The time required for the reaction is different depending on general conditions such as the amount of water to be added, the reaction temperature and the like. For example, in one embodiment when the reaction is carried out at about 25° C. using sodium tetraborate decahydrate containing water of crystallization, and the amount of water is 1 mole based on 1 mole of the titanium tetraalkoxide compound, the time required for stirring is preferably about 48 hours because much higher enantioselectivity is exhibited in the asymmetric cyanation reaction. When the amount of water is about 1.25 mole at 25° C. based on about 1 mole of the titanium tetraalkoxide compound, the reaction can be carried out by stirring for about 20 hours. Next, the optically active ligand is added. The titanium compound can also be produced by mixing the titanium oxoalkoxide compound represented by the above general formula (I) with the optically active ligand represented by the above general formula (II).

The optically active ligand may be added in such an amount to the titanium tetraalkoxide compound with water or the titanium oxoalkoxide so that a mole fraction of titanium to the optically active ligand of about 0.5≦Ti/ligand≦4 is obtained. In one embodiment of the present invention, the mole fraction of titanium to optically active ligand may be about 1≦Ti/ligand≦about 3, such as Ti/ligand=2. Thus, the ratio of titanium to ligand may be shifted to titanium, i.e. in the process of the present invention good yields may still be obtained if the catalyst composition contains more titanium than chiral ligand. Further, the optically active ligand may be dissolved in a solvent or may be added as it is without being dissolved. When a solvent is used, the solvent can be the same solvent as or different from the solvent used in the above step of adding water. When a solvent is newly added, the amount thereof may be from about 1 to about 5.000 ml and preferably from about 1 to about 500 ml, based on 1 mmole of the titanium atom. At this time, the reaction temperature is not particularly limited, but the compound can be usually produced by stirring at about room temperature, for example, from 15 to 30° C. for about 5 minutes to about 1 hour or about 30 minutes to about 1 hour. The production of the titanium compound of the present invention is preferably carried out under a dry inert gas atmosphere. Examples of the inert gas include nitrogen, argon, helium and the like. Subsequent to stirring of the reaction mixture the titanium compound of the present invention is obtained. At that time, in order to smoothly progress the reaction, a solvent may be used. The solvent in use dissolves any one of the titanium oxoalkoxide compound or optically active ligand, or both of them to smoothly progress the reaction. Examples of the solvent include halogenated hydrocarbon solvents such as dichloromethane, chloroform and the like; halogenated aromatic hydrocarbon solvents such as fluorobenzene, trifluoromethylbenzene and the like; aromatic hydrocarbon solvents such as toluene, xylene and the like; ester solvents such as ethyl acetate and the like; ester solvents such as ethyl acetate and the like; and ether solvents such as tetrahydrofuran, dioxane, diethyl ether, dimethoxyethane and the like. In one embodiment, halogenated hydrocarbon solvents or aromatic hydrocarbon solvents may be used. In a further embodiment, acetonitrile may also be used. In a still further embodiment, mixtures of the above solvents may also be used.

The total amount of the solvent used may be from about 1 to about 5.000 ml or from about 10 to about 500 ml, based on 1 mmole of the titanium atom in the titanium oxoalkoxide compound. The temperature at this time is not particularly limited, but the reaction can be usually carried out at about room temperature, for example, from 15 to 30° C. The time required for preparing a catalyst may be about 5 minutes to about 1 hour or about 30 minutes to about 1 hour.

In a further embodiment, when a catalyst is prepared by mixing the titanium oxoalkoxide compound with the optically active ligand in a solvent, alcohols can also be added. Examples of the alcohols to be added include, but are not limited to, an aliphatic alcohol and an aromatic alcohol, each of which may be optionally substituted, and one kind or two or more kinds may be mixed, prior to use. As the aliphatic alcohol, a linear, branched or cyclic alkyl alcohol having 1 to 10 carbon atoms may be used. Examples include, but are not limited to, methanol, ethanol, n-propanol, isopropanol, n-butanol, sec-butanol, tert-butanol, cyclopentyl alcohol, cyclohexyl alcohol and the like.

The aforementioned linear, branched or cyclic alkyl alcohol may be optionally substituted as mentioned above. Examples of substituted alcohols may include, but are not limited to, chloromethanol, 2-chloroethanol, trifluoromethanol, 2,2,2-trifluoroethanol, perfluoroethanol, perfluorohexyl alcohol and the like. As the aromatic alcohol, an aryl alcohol having 6 to 20 carbon atoms may be used, and examples thereof may include, but are not limited to, phenol, naphthol and the like. The aryl alcohol may be optionally substituted. Examples thereof may include, but are not limited to, pentafluorophenol, dimethylphenol, trimethylphenol, isopropylphenol, diisopropylphenol, tert-butylphenol, di-tert-butylphenol and the like.

When a catalyst is prepared by adding these alcohols, the amount thereof is from about 0.5 to about 20 moles or from about 1 to about 10 moles, based on 1 mole of the titanium atom of the above titanium compound. Further, these alcohols may be added at the time of producing the aforementioned titanium compound. Due to this, in the asymmetric cyanation reaction, high reactivity and high optical yield can be obtained with good reproducibility. The titanium compound produced as above can be used for the asymmetric catalytic reaction as it is without carrying out a special purification operation. In particular, the compound is suitable for the asymmetric cyanation reaction of aldehyde or asymmetric ketone of the present invention.

In the method of the present invention, the aldehydes or ketones to be used as a starting material are not particularly limited as far as they are prochiral compounds having a carbonyl group in a molecule, and can be suitably selected corresponding to the desired optically active cyanohydrins.

The method of the present invention is particularly suitable when producing corresponding optically active cyanohydrins with a carbonyl compound represented by the general formula (V)

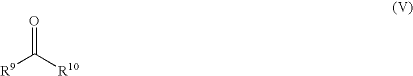

as a starting material.

In the above general formula (V), R⁹ and R¹⁰ are different groups, and each represents a hydrogen atom, an alkyl group, an alkenyl group, an alkynyl group, an aryl group, an aromatic heterocyclic group or a non-aromatic heterocyclic group, each of which may be optionally substituted. Furthermore, R⁹ and R¹⁰ may be optionally linked together to form a ring.

The aldehyde used in the method of the present invention may be, for example, an aliphatic aldehyde, an α,β-unsaturated aldehyde or an aromatic aldehyde. Examples of aldehydes that can be used as a starting material in the method of the present invention include, but are not limited to, propionaldehyde, butylaldehyde, valeraldehyde, isovaleraldehyde, hexaaldehyde, heptaldehyde, octylaldehyde, nonylaldehyde, decylaldehyde, isobutylaldehyde, 2-methylbutylaldehyde, 2-ethylbutylaldehyde, 2-ethylhexanal, pivalaldehyde, 2,2-dimethylpentanal, cyclopropanecarboaldehyde, cyclohexanecarboaldehyde, phenylacetaldehyde, (4-methoxyphenyl)acetaldehyde, 3-phenylpropionaldehyde, benzyloxyacetaldehyde, crotonaldehyde, 3-methylcrotonaldehyde, methacrolein, trans-2-hexenal, trans-cinnamaldehyde, benzaldehyde, o-, m- or p-tolylaldehyde, 2,4,6-trimethylbenzaldehyde, 4-biphenylcarboaldehyde, o-, m- or p-fluorobenzaldehyde, o-, m- or p-chlorobenzaldehyde, o-, m- or p-bromobenzaldehyde, 2,3-, 2,4- or 3,4-dichlorobenzaldehyde, 4-(trifluoromethyl)benzaldehyde, 3- or 4-hydroxybenzaldehyde, 3,4-dihydroxybenzaldehyde, o-, m- or p-anisaldehyde, 4-methoxybenzaldehyde, 3,4-dimethoxybenzaldehyde, 3,4-(methylenedioxy)benzaldehyde, m- or p-phenoxybenzaldehyde, m- or p-benzyloxybenzaldehyde, 2,2-dimethylchromane-6-carboaldehyde, 1- or 2-naphthaldehyde, 2- or 3-furancarboaldehyde, 2- or 3-thiophenecarboaldehyde, 1-benzothiophene-3-carboaldehyde, N-methylpyrrole-2-carboaldehyde, 1-methylindole-3-carboaldehyde, 2-, 3- or 4-pyridinecarboaldehyde, 2-furaldehyde, tiglic aldehyde, trans-2-hexanal and the like.

In a further embodiment of the present invention, examples of unsymmetrical ketones which can be used as a starting material in the method of the present invention include, but are not limited to, 2-butanone, 2-pentanone, 2-hexanone, 2-heptanone, 2-octanone, isopropylmethyl ketone, cyclopentylmethyl ketone, cyclohexylmethyl ketone, phenylacetone, p-methoxyphenylacetone, 4-phenylbutane-2-on, cyclohexylbenzyl ketone, acetophenone, o-, m- or p-methylacetophenone, 4-acetylbiphenyl, o-, m- or p-fluoroacetophenone, o-, m- or p-chloroacetophenone, o-, m- or p-bromoacetophenone, 2′,3′-, 2′,4′- or 3′,4′-dichloroacetophenone, m- or p-hydroxyacetophenone, 3′,4′-dihydroxyacetophenone, o-, m- or p-methoxyacetophenone, 3′,4′-dimethoxyacetophenone, m- or p-phenoxyacetophenone, 3′,4′-diphenoxyacetophenone, m- or p-benzyloxyacetophenone, 3′,4′-dibenzyloxyacetophenone, 2-chloroacetophenone, 2-bromoacetophenone, propiophenone, 2-methylpropiophenone, 3-chloropropiophenone, butyrophenone, phenylcyclopropyl ketone, phenylcyclobutyl ketone, phenylcyclopentyl ketone, phenylcyclohexyl ketone, 1- or 2-acenaphthone, chalcone, 1-indanone, 1- or 2-tetralon, 4-chromanone, trans-4-phenyl-3-butene-2-on, 2- or 3-acetylfuran, 2- or 3-acetylthiophene, 2-, 3- or 4-acetylpyridine and the like.

The amount of aldehyde or asymmetric ketone used in the present process is not critical. However, it should be noted that an increase of concentration thereof might result in an increase of reaction rate but also in a decrease in enantioselectivity (cf. Example 6).

In the method of the present invention, a cyanating agent may be used, which may be at least one kind selected from hydrogen cyanide, acetone cyanohydrin, cyanoformate ester, acetyl cyanide, dialkylcyanophosphates, trialkylsilyl cyanide, potassium cyanide-acetic acid and potassium cyanide-acetic anhydride. The cyanating agent may be used in an amount of about 1 to about 3 moles or in an amount of about 1.05 to about 2.5 moles or in an amount of about 1.5 to about 2.5 moles, based on the amount of aldehyde or asymmetrical ketone, i.e. based on 1 mole of the aldehyde or asymmetrical ketone.

In one illustrative embodiment of the present invention, the cyanating agent is methyl cyanoformate or ethyl cyanoformate. Cyanoformate esters are attractive as alternative cyanating agents because they are cheaper than TMSCN, less toxic, less susceptible to hydrolysis and hence easier to handle. In addition, the resultant cyanohydrin carbonates are more stable and less prone to hydrolysis as compared to cyanohydrin TMS ethers.

The cyanation reaction of the present invention is additionally catalyzed by Lewis bases. A Lewis base is any molecule or ion that can form a new coordinate covalent bond, by donating a pair of electrons, i.e. any molecule with an electron lone pair in a bonding orbital may act as a Lewis base. Suitable Lewis bases useful for the activation in the present invention may include, but are not limited to, NR¹¹R¹²R¹³, O═NR¹¹R¹²R¹³, dialkylaminopyridine, diarylaminopyridine and N,N,N,N-tetramethylethylenediamine, wherein R¹¹, R¹² and R¹³ are independently selected from the group consisting of hydrogen, alkyl and aryl. In one embodiment of the present invention, the Lewis base may be triethylamine or 4-dimethylaminopyridine. It is also possible to use chiral Lewis bases in the reaction of the present invention. Examples of such chiral Lewis base may include, but are not limited to, (−)-cinchonidine, (+)-cinchonine and (−)-sparteine.

The Lewis base may be used in any suitable amount that is able to efficiently promote the cyanation reaction. The Lewis base may be used in an amount of about 1 to about 10 mol % with respect to the amount of the aldehyde or asymmetrical ketone used in the process of the present invention. In one embodiment of the present invention, about 1 to about 5 mol %, such as about 1 to about 3 mol % of the Lewis base with respect to the amount of aldehyde or asymmetrical ketone may be used. The use of suitable Lewis bases leads to an increased reactivity and thus to a higher conversion of the aldehydes or asymmetrical ketones to the respective cyanohydrins with an increased enantiomeric excess (ee). It was for example found here that the use of 4-dimethylaminopyridine (DMAP) is able to yield in a conversion of 85% and an enantiomeric excess of 72% ee in illustrative reactions. It has also been found that no cyanation with aldehydes occurs in the absence of the Lewis base additive. Further illustrative results can be taken from Example 21. It should be further noted, that, for example, a DMAP loading near or even above 10 mol % would result in a faster reaction rate but lower enantioselectivity (cf. Example 5).

Furthermore, the amount of the aforementioned optically active catalyst, i.e. the titanium compound, to be used in the method of the present invention may be in the range from about 0.01 to about 30 mole % or from about 1 to about 10.0 mole % in terms of the titanium atom with respect to the amount of aldehyde or unsymmetrical ketone, i.e. based on 1 mole of aldehyde or asymmetrical ketone. In one embodiment of the present invention about 3 to about 5 mol % of the optically active catalyst in terms of the titanium atom with respect to the amount of the aldehyde or unsymmetrical ketone is used.

In one embodiment of the method of the present invention, a solvent may be used during the preparation process. Examples of the solvent include, but are not limited to, halogenated hydrocarbon solvents such as dichloromethane, chloroform and the like; halogenated aromatic hydrocarbon solvents such as chlorobenzene, o-dichlorobenzene, fluorobenzene, trifluoromethylbenzene and the like; aromatic hydrocarbon solvents such as toluene, xylene and the like; ester solvents such as ethyl acetate and the like; and ether solvents such as tetrahydrofuran, dioxane, diethyl ether, dimethoxyethane, cyclopentylmethyl ether and the like. In one embodiment of the present invention, halogenated hydrocarbon solvents or aromatic hydrocarbon solvents are used. In a further embodiment, acetonitrile may also be used. Furthermore, these solvents can be used singly or in combination as a mixed solvent. The total amount of the solvent used may be from about 1 to about 20 ml, for example from about 5 to about 10 ml, based on 1 mmole of the substrate aldehyde or asymmetrical ketone.

The reaction of the present invention can be carried out by adding an appropriate solvent to a solution of the titanium compound produced according to the present invention, and stirring the mixture at room temperature for a suitable time, for example, about 30 minutes. Then a substrate aldehyde, cyanating agent and Lewis base are added in order, and for the reaction the resulting solution is stirred at about −10 to 40° C. or any temperature as indicated below for a suitable time, for example, for about 30 minutes to about 24 hours, for example for about 1 hour to about 24 hours or for about 1 hour to about 20 hours.

As said, the reaction of the aldehyde or asymmetrical ketone with the cyanating agent in the presence of a Lewis base and a titanium compound may be carried out in a temperature range of from about −10° C. to about 40° C. The temperature can be chosen depending on the compounds used in the preparation process, i.e. the aldehyde or asymmetrical ketone, the Lewis base, the cyanating agent and the titanium compound. In one embodiment of the present invention, the reaction may be carried out at room temperature, for example at a temperature between about 15° C. and about 30° C. or at a temperature between 20° C. to about 25° C.

With the process of the present invention, the preparation of cyanohydrin derivatives is improved over the prior art in various aspects. First, a higher conversion of the respective aldehydes or asymmetrical ketones may be achieved with less catalyst. Second, a higher enantioselectivity can be obtained within a shorter period of time. Third, the reaction may even be carried out without the need for cooling but at room temperature or elevated temperature. With the process of the present invention a conversion of the aldehyde or asymmetrical ketone of at least up to 85% may be obtained. In further embodiments, a conversion of at least up to 95% may be achieved. However, it is of course also possible to have less conversion when the respective conversion may occur within a shorter period of time compared to known process in the prior art.

As mentioned, the process of the present invention typically can lead to an improved enantioselectivity of the conversion reaction. “Enantioselectivity” is the preferential formation in a chemical reaction of one enantiomer over another. It is quantitatively expressed by the enantiomeric excess. With the process of the present invention an enantiomeric excess of >about 65% ee may be obtained. In one embodiment the cyanohydrin may be obtained in an enantiomeric excess of>about 80% ee. The values of respective reactions can be taken from the Examples below. It should be noted, that there is a positive and moderate correlation between ee of the ligand and ee of the product, i.e. an increased enantiomeric purity of the used educts (ligands) will lead to an increase ee of the product (cyanohydrin). It should be further noted that it is possible to obtain an ee<65% for specific aldehydes or ketones, however, this ee will still be above the ee obtainable by the prior art processes.

In the present invention, asymmetric cyanation of aldehydes with cyanoformate ester at room temperature has been achieved with high conversions and enantioselectivities particularly for the aliphatic aldehydes. High conversions and moderate to good enantioselectivities have also been obtained for the aromatic and α,β-unsaturated aldehydes.

The asymmetric cyanation reaction of the present invention can be used for the production of optically active cyanohydrins that are compounds useful in the fields of medical supplies and agrichemical chemicals, and functional materials.

### EXAMPLES

The following experimental examples are provided to further illustrate the present invention and are not intended to be limiting to the scope of the invention.

The product of the asymmetric cyanation reaction was identified by mass spectrometry (ESI-MS using Shimadzu LC-20AT Tandem Mass Spectrometer) and by comparison of the ¹H NMR spectrum (recorded on a Bruker 400 UltraShield instrument with CDCl₃ as solvent) with that reported in the literature. The conversion and yield of the asymmetric cyanation reaction was obtained by using gas chromatography (Agilent 6890N) on a CHIRALDEX G-TA chiral column, with dodecane as the internal standard. The absolute configuration of the product was determined by comparison with literature reported specific rotation (using a Jasco P-1030 polarimeter) for the cyanohydrin O-carbonates or the corresponding cyanohydrin O-acetates or cyanohydrin O-TMS ethers. Titanium tetra-n-butoxide (from Fluka), 4-Dimethylaminopyridine (from Fluka), sodium tetraborate decahydrate (from Kanto) and anhydrous dichloromethane (from Kanto) were used directly without further purification. Ethyl cyanoformate (from Acros Organics) and the aldehydes were preferably distilled prior to use. All the reactions were carried out under a nitrogen atmosphere.

### Example 1

### Preparation of a Solution of a Partial Hydrolysate of Titanium tetra-n-butoxide

a) Titanium tetra-n-butoxide (0.170 g, 0.500 mmol) and sodium tetraborate decahydrate (0.0191 g, 0.0500 mmol) were stirred in anhydrous dichloromethane (3 ml) at 200 rpm for 48 h at room temperature. The mixture was filtered through a 0.2 μm PTFE membrane into a 10.0 ml volumetric flask. It was diluted to the mark with anhydrous dichloromethane. Then the partially hydrolyzed titanium precursor solution (0.050 M) was transferred to a sample bottle and stirred at room temperature.

b.) Titanium tetra-n-butoxide (0.170 g, 0.500 mmol) and sodium tetraborate decahydrate (0.0238 g, 0.0625 mmol) were stirred in anhydrous dichloromethane (3 ml) at 200 rpm for 20 h at room temperature. The mixture was filtered through a 0.2 μm PTFE membrane into a 10.0 ml volumetric flask. It was diluted to the mark with anhydrous dichloromethane. Then the partially hydrolyzed titanium precursor solution (0.050 M) was transferred to a sample bottle and stirred at room temperature.

### Example 2

### Preparation of Titanium Catalyst

The partial hydrolysate of titanium tetra-n-butoxide prepared from example 1 (0.050 M, 1.0 ml, 0.050 mmol) was added to a solution of the chiral ligand, (S)-2-(N-3,5-di-tert-butylsalicylidene)amino-3-methyl-1-butanol (0.0160 g, 0.0501 mmol) in anhydrous dichloromethane (2.5 ml) in a 5.0 ml volumetric flask. It was diluted to the mark with anhydrous dichloromethane. Then the titanium catalyst solution (0.010 M) was transferred to a sample bottle and stirred at 1000 rpm for 30 mins at room temperature.

### Example 3

### General Procedure for Asymmetric Cyanation

Of Aliphatic Aldehydes:

Titanium catalyst solution prepared from example 2 (0.010 M, 1.0 ml, 0.010 mmol) was added to anhydrous dichloromethane (1.0 ml). Aliphatic aldehyde (0.20 mmol) was added to the titanium catalyst followed by ethyl cyanoformate (0.040 ml, 0.40 mmol). After that freshly prepared 4-dimethylaminopyridine solution in anhydrous dichloromethane (0.20 M, 0.020 ml, 0.0040 mmol) was added to the mixture and stirred at room temperature for 24 h. Dodecane (0.034 ml, 0.15 mmol) was added to the mixture as internal standard. The mixture was analyzed by GC. The crude product can be subsequently purified by silica column chromatography with hexane-ethyl acetate as eluent.

### Of Aromatic Aldehydes:

The procedures were similar to that for aliphatic aldehydes except that additional 1.0 ml of anhydrous dichloromethane was not required and only 0.0020 mmol of 4-dimethylaminopyridine was added.

### Of α,β-Unsaturated Aldehydes:

The procedures were similar to that for aromatic aldehydes except that 0.0040 mmol of 4-dimethylaminopyridine was added.

### Example 4

### Effect of Lewis Base

The effect of the use of Lewis base additives in the preparation process of the present invention can be taken from Table 1 below, which shows the results of the preparation process as given for specific Lewis bases. The results indicate that the use of Lewis base additives can improve the reactivity of the claimed process. For example, the use of 4-dimethylaminopyridine leads to a conversion of 85% and an enantiomeric excess of 72% ee. It is also shown that no cyanation with aldehydes occur in the absence of the additive.

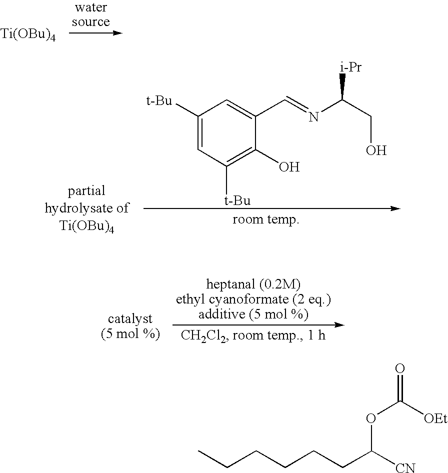

TABLE 1

| | | Enantiomeric |
|---|---|---|
| | | excess, ee |
| | Conversion | (%) [absolute |
| Additive | (%) | configuration] |
| No additive | 0 | — |
| Triethylamine | 98 | 59 [S] |
| 4-Dimethylaminopyridine (DMAP) | 85 | 72 [S] |
| Diisopropylamine | 71 | 48 [S] |
| N,N,N,N-Tetramethylethylenediamine | 72 | 73 [S] |
| (TMEDA) | | |

### Example 5

### Effect of the Amount of DMAP, Ethyl Cyanoformate and Ti-Catalyst

This Example shows the effect of DMAP (Lewis base), ethyl cyanoformate and the Ti-catalyst in the preparation process of the present invention. It can be taken from Table 2 below that a catalyst loading >5 mol % does not result in significant increase in enantioselectivity, whereas catalyst loading <5 mol % results in a decrease in reaction rate and enantioselectivity. An increase in DMAP loading results in a faster reaction rate but a lower enantioselectivity. Further, a decrease in the amount of ethyl cyanoformate results in a slower reaction rate with a lower enantioselectivity.

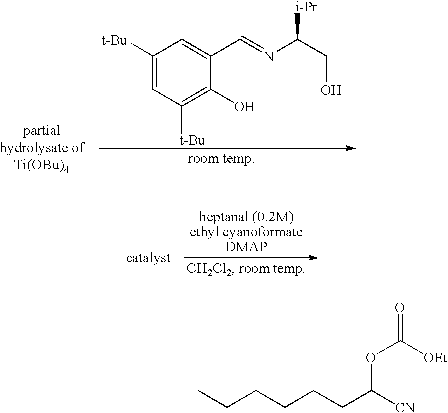

TABLE 2

| | | Ethyl | | | ee (%) |
|---|---|---|---|---|---|
| Catalyst | DMAP | cyanoformate | Time | Conversion | [absolute |
| (mol %) | (mol %) | (eq.) | (h) | (%) | configuration] |
| 0 | 5 | 2 | 2 | 29 | 0 |
| 5 | 10 | 2 | 2 | 98 | 52 [S] |
| 5 | 5 | 2 | 2 | 97 | 71 [S] |
| 5 | 2 | 2 | 6 | 96 | 80 [S] |
| 5 | 1 | 2 | 6 | 43 | 83 [S] |
| 10 | 10 | 2 | 2 | 96 | 58 [S] |
| 10 | 5 | 2 | 2 | 94 | 75 [S] |
| 10 | 2 | 2 | 2 | 89 | 83 [S] |
| 3 | 1 | 2 | 24 | 90 | 37 [S] |
| 2 | 1 | 2 | 24 | 79 | 14 [S] |
| 1 | 0.5 | 2 | 24 | <1 | — |
| 5 | 5 | 1.5 | 2 | 97 | 68 [S] |
| 5 | 2 | 1.5 | 24 | 98 | 76 [S] |

### Example 6

### Effect of Substrate Concentration

In this Example, the effect of changing the substrate concentration in the preparation process was tested. Heptanal was used as test compound. As can be taken from Table 3, an increase in substrate concentration results in increase of reaction rate but decrease in enantioselectivity. Thus, it appears that the concentration of the compounds used in the process has an influence on obtaining sufficient enantioselectivity.

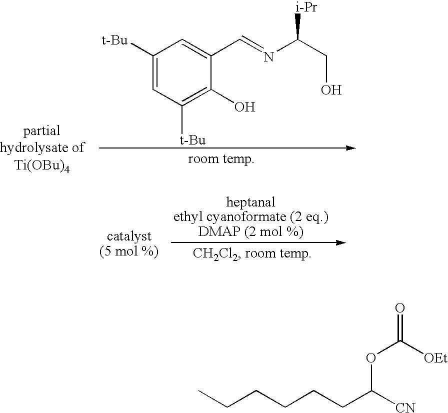

TABLE 3

| | | | ee (%) [absolute |
|---|---|---|---|
| Conc. of heptanal (M) | Time (h) | Conversion (%) | configuration] |
| 1.0 | 2 | 99 | 9 [S] |
| 0.5 | 2 | 99 | 28 [S] |
| 0.2 | 6 | 96 | 80 [S] |
| 0.1 | 24 | 99 | 84 [S] |
| 0.05 | 24 | 87 | 84 [S] |

### Example 7

### Effect of Reaction Temperature

As already stated above, it is possible to conduct the cyanation process of the present invention at evaluated temperatures compared to the processes of the prior art. As can be taken from Table 4, the reaction at 0° C. or even at room temperature (25° C.) results in high conversion and excellent enantiomeric excess for the test compound heptanal. Lower reaction temperature results in decrease in enantioselectivity.

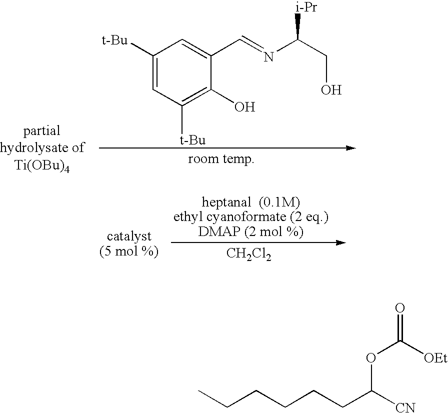

TABLE 4

| | | | ee (%)[absolute |
|---|---|---|---|
| Temp. (° C.) | Time (h) | Conversion (%) | configuration] |
| 40 | 20 | 97 | 80 [S] |
| 25 | 24 | 99 | 84 [S] |
| 0 | 24 | 99 | 78 [S] |
| −20 | 24 | 99 | 41 [S] |
| −40 | 24 | 87 | 3 [S] |

### Example 8

In this Example, heptanal was used as a model substrate for aliphatic aldehydes, and the optimized reaction conditions are applied for the asymmetric cyanation of aliphatic aldehydes. The results in Table 5 show that the cyanation process of the present invention conducted at room temperature results in almost complete conversion of the aliphatic aldehydes with high amounts of enantiomeric excess.

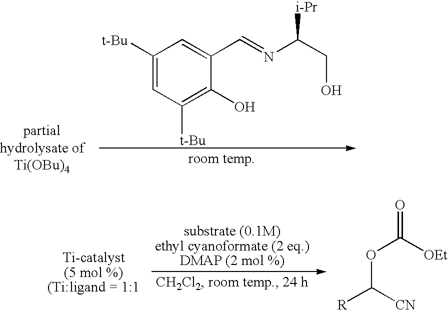

TABLE 5

| | | ee (%) [absolute |
|---|---|---|
| Substrate | Conversion (%) | configuration] |
| Heptanal | 99 | 84 [S] |
| Isovaleraldehyde | 99 | 86 |
| Isobutyraldehyde | 99 | 93 [S] |
| cyclohexane carboxaldehyde | 99 | 94 [S] |
| Pivalaldehyde | 99 | 94 [S] |

### Example 9

### Asymmetric Cyanation of Aromatic Aldehydes

Benzaldehyde was used as a model substrate for aromatic aldehydes, and the optimized reaction conditions were applied for the asymmetric cyanation of aromatic aldehydes. The optimized conditions for the asymmetric cyanation of benzaldehyde were obtained by screening of the various parameters.

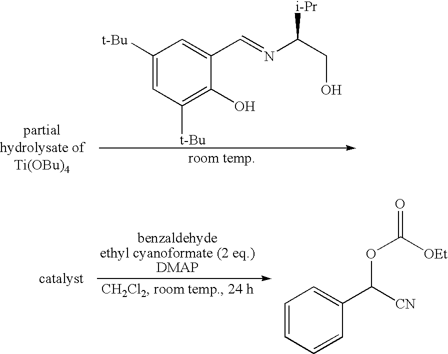

TABLE 6

| Catalyst | DMAP | Conc. of | | ee (%) [absolute |
|---|---|---|---|---|
| (mol %) | (mol %) | benzaldehyde (M) | Conversion (%) | configuration] |
| 5 | 5 | 0.2 | 99 | 56 [S] |
| 5 | 2 | 0.2 | 99 | 71 [S] |
| 5 | 1 | 0.2 | 99 | 79 [S] |
| 3 | 1 | 0.2 | 95 | 78 [S] |
| 2 | 1 | 0.2 | 78 | 78 [S] |
| 10 | 3 | 0.1 | 99 | 72 [S] |
| 10 | 2 | 0.1 | 99 | 70 [S] |
| 10 | 1 | 0.1 | 73 | 61 [S] |
| 5 | 2 | 0.1 | 98 | 73 [S] |
| 5 | 1 | 0.1 | 53 | 74 [S] |
| 3 | 1 | 0.1 | 43 | 76 [S] |

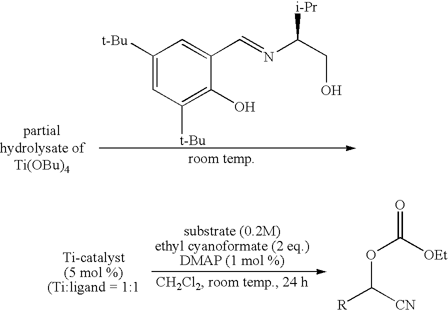

TABLE 7

| | | ee (%) [absolute |
|---|---|---|
| Substrate | Conversion (%) | configuration] |
| Benzaldehyde | 99 | 79 [S] |
| 2-fluorobenzaldehyde | 99 | 79 [S] |
| 4-fluorobenzaldehyde | 95 | 76 |
| 4-methoxybenzaldehyde | 97 | 70 [S] |
| 2-furaldehyde | 94 | 70 [R] |

### Example 10

### Asymmetric Cyanation of α,β-Unsaturated Aldehydes

Also, α,β-unsaturated aldehydes were tested according to the chemical equation below. The results indicate that this type of aldehydes may also be converted with high ee to the respective cyanohydrins.

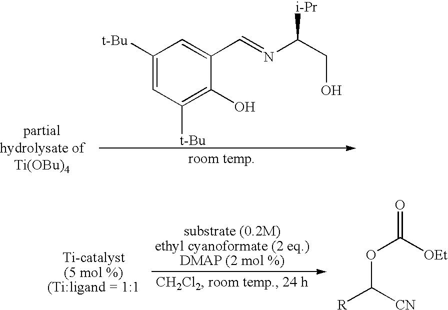

TABLE 8

| | | ee (%) [absolute |
|---|---|---|
| Substrate | Conversion (%) | configuration] |
| tiglic aldehyde | 99 | 70 [S] |
| trans-2-hexenal | 99 | 67 [S] |

### Example 11

### Use of Less Chiral Ligand

In this Example, the effect of the use of less chiral ligand was tested. Although the preferred Ti:ligand ratio is about 1:1, a reduction in the amount of the chiral ligand used to a Ti:ligand ratio of about 2:1 can still result in similar catalyst performance in terms of conversion and enantioselectivity, provided that a suitable partial hydrolysate of titanium tetrabutoxide is used. The results in Table 10 confirm this result.

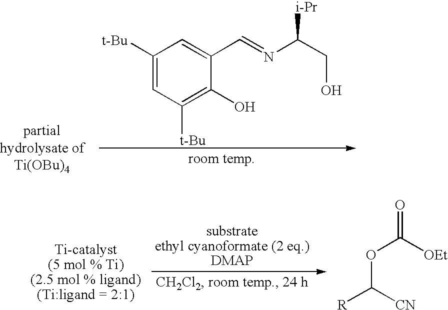

TABLE 9

| | | Conc. of | | | ee (%)^{b} |
|---|---|---|---|---|---|
| | DMAP | substrate | Conversion | Yield | [absolute |
| Substrate | (mol %) | (M) | (%)^{a} | (%)^{a} | configuration]^{c} |
| Heptanal | 2 | 0.1 | 99 | 86 | 85 [S] |
| Isovaleraldehyde | 2 | 0.1 | 99 | 98 | 86 [ND]^{d} |
| Isobutyraldehyde | 2 | 0.1 | 99 | 90 | 91-93 [S] |
| Cyclohexane | 2 | 0.1 | 99 | 91-99 | 92-95 [S] |
| carboxaldehyde | | | | | |
| Pivalaldehyde | 2 | 0.1 | 99 | 99 | 91-93 [S] |
| Benzaldehyde | 1 | 0.2 | 99 | 98 | 78 [S] |
| 2-Fluorobenzaldehyde | 1 | 0.2 | 99 | 94-97 | 79-80 [S] |
| 4-Fluorobenzaldehyde | 1 | 0.2 | 95 | 89 | 76 [ND]^{d} |
| 4-Methoxybenzaldehyde | 1 | 0.2 | 97 | 90 | 69 [S] |
| 2-Furaldehyde | 1 | 0.2 | 92 | 92 | 71 [R] |
| Tiglic aldehyde | 2 | 0.2 | 98 | 89 | 71 [S] |
| Trans-2-hexenal | 2 | 0.2 | 99 | 91 | 68 [S] |

^{a}determined by GC analysis with dodecane as internal standard
^{b}determined by GC analysis on a CHIRALDEX G-TA chiral column
^{c}absolute configuration were assigned by comparison with reported specific rotation for the cyanohydrin O-carbonates or the corresponding cyanohydrin O-acetates or cyanohydrin O-TMS ethers
^{d}ND—not determined

### REFERENCES

The following references are cited in the present application:
1. Yoshinaga, K.; Nagata, T.; Miyazoe, S. PCT, WO2006/041000A1.
2. Lundgren S.; Wingstrand, E.; Penhoat, M.; Moberg, C. J. Am. Chem. Soc. 2005, 127, 11592.
3. Wingstrand, E.; Lundgren S.; Penhoat, M.; Moberg, C. Pure Appl. Chem 2006, 78, 409.
4. Belokon, Y. N.; Ishibashi, E.; Nomura, H.; North, M. Chem. Commun. 2006, 1775.
5. Belokon, Y. N.; Blacker, A. J.; Clutterbuck, L. A.; North, M. Org. Lett. 2003, 5, 4505.
6. Belokon, Y. N.; Blacker, A. J.; Carta, P.; Clutterbuck, L. A.; North, M. Tetrahedron 2004, 60, 10433.
7. Casas, J.; Baeza, A.; Sansano, J. M.; Nájera, C.; Saá, J. M. Tetrahedron: Asymmetry 2003, 14, 197.
8. Baeza, A.; Casas, J.; Nájera, C.; Sansano, J. M.; Saá, J. M. Eur. J. Org. Chem. 2006, 1949.
9. Tian, J.; Yamagiwa, N.; Matsunaga, S.; Shibasaki, M. Angew. Chem. Int. Ed. 2002, 41, 3636.
10. Yamagiwa, N.; Tian, J.; Matsunaga, S.; Shibasaki, M. J. Am. Chem. Soc. 2005, 127, 3413.
11. Gou, S.; Chen, X.; Xiong, Y.; Feng, X. J. Org. Chem. 2006, 71, 5732.
12. Day, V. W.; Eberspacher, T. A.; Klemperer, W. G.; Park, C. W.; Rosenberg, F. S., J. Am. Chem. Soc. 1991, 113, 8190.
13. Day, V. W.; Eberspacher, T. A.; Chen, Y.; Hao, J.; Klemperer, W. G., Inorg. Chim. Acta, 1995, 229, 391.
14. Steunou, N.; Kickelbick, G.; Boubekeur, K.; Sanchez, C. J. Chem. Soc. Dalton Trans., 1999, 3653

1. A method of producing an optically active cyanohydrin derivative, which comprises reacting an aldehyde or an unsymmetrical ketone with a cyanating agent in the presence of a Lewis base and a titanium compound produced from a partial hydrolysate of titanium tetraalkoxide and an optically active ligand represented by the general formula (II) or a titanium oxoalkoxide compound represented by the general formula (I) and an optically active ligand represented by the general formula (II),
[TiₓO_{y}](OR¹)_{4x-2y} (I)
wherein R¹ is an optionally substituted alkyl group or an optionally substituted aryl group; x is an integer of not less than 2; y is an integer of not less than 1; and y/x satisfies 0.1<y/x≦1.5, 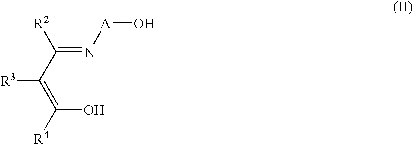
wherein R², R³ and R⁴ are independently a hydrogen atom, an alkyl group, an alkenyl group, an aryl group, an aromatic heterocyclic group, a non-aromatic heterocyclic group, an acyl group, an alkoxycarbonyl group or an aryloxycarbonyl group, each of which may be optionally substituent, two or more of R², R³ and R⁴ may be linked together to form a ring, and the ring may have a substituent; and A represents a hydrocarbon containing group with three or more carbon atoms having at least one asymmetric carbon atom or axial asymmetry.

2. The method according to claim 1, wherein the titanium compound produced from a partial hydrolysate of titanium tetraalkoxide is obtained from reaction of about 1 mole of titanium tetraalkoxide with less than about 2 moles of water and an optically active ligand represented by the general formula (II).

3. The method according to claim 1, wherein the hydrocarbon containing group A is a hydrocarbon containing group represented by any one of the general formulae (A-1), (A-2) or (A-3), 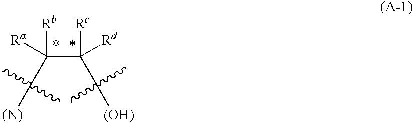
wherein R^{a}, R^{b}, R^{c} and R^{d} are each independently a hydrogen atom, an alkyl group, an aryl group, an alkoxycarbonyl group, an aryloxycarbonyl group or an aminocarbonyl group, each of which may be optionally substituted, two or more of R^{a}, R^{b}, R^{c} and R^{d} may be linked together to form a ring, and the ring may be optionally substituted; at least one of R^{a}, R^{b}, R^{c} and R^{d} is not hydrogen; both or at least one of the carbon atoms indicated as * become an asymmetric centre; and parts indicated as (N) and (OH) do not belong to A, and represent a nitrogen atom and a hydroxyl group corresponding to those in said general formula (I) to which A is bonded, 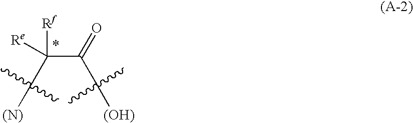
wherein R^{e} and R^{f} are each independently a hydrogen atom, an alkyl group or an aryl group, each of which may be optionally substituted; R^{e} and R^{f} are different substituents and * represents an asymmetric carbon atom; and parts indicated as (N) and (OH) represent the same as those in the general formula (A-1), or 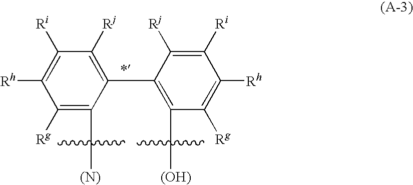
wherein R^{g}, R^{h}, Rⁱ and R^{j} are independently a hydrogen atom, a halogen atom, an alkyl group, an aryl group or an alkoxy group, each of which may be optionally substituted, Rⁱ and R^{j} on the same benzene ring may be linked or condensed together to form a ring, and *′ represents an axial symmetry; and parts indicated as (N) and (OH) represent the same as those in the general formula (A-1).

4. The method according to claim 1, wherein the optically active ligand is represented by the general formula (III) 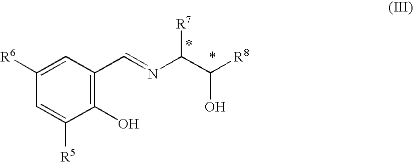
wherein
R⁵, R⁶, R⁷ and R⁸ are independently a hydrogen atom, a halogen atom, an alkyl group, an alkenyl group, an alkynyl group, an aryl group, a aromatic heterocyclic group, a non-aromatic heterocyclic group, an alkoxycarbonyl group, an aryloxycarbonyl group, each of which may be optionally substituted, cyano, nitro, OH, an alkoxy group, an amino group, a silyl group or a siloxy group; wherein at least one of R⁷ and R⁸ is not hydrogen, and wherein R⁷ and R⁸ together may form an optionally substituted ring having 4 to 8 carbon atoms, both or at least one of the carbon atoms indicated as * become an asymmetric center.

5. The method according to claim 4, wherein the optically active ligand is selected from the group consisting of:

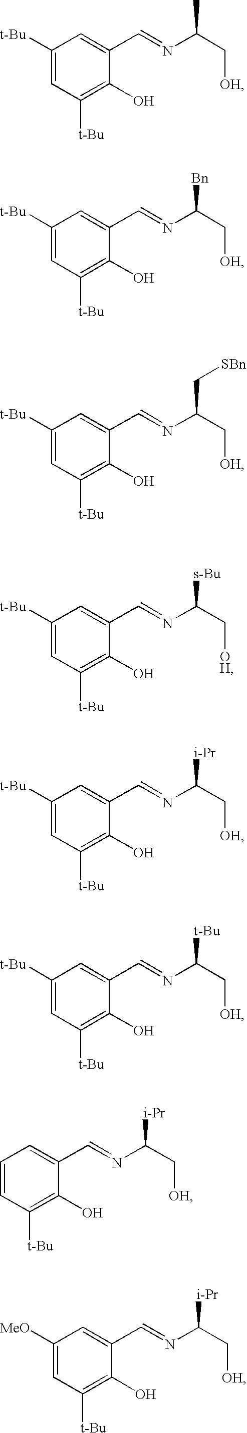

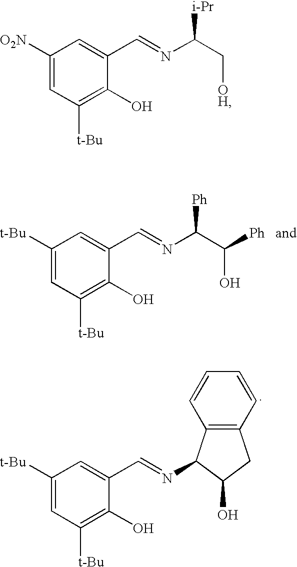

6. The method according to claim 1, wherein the optically active ligand
is

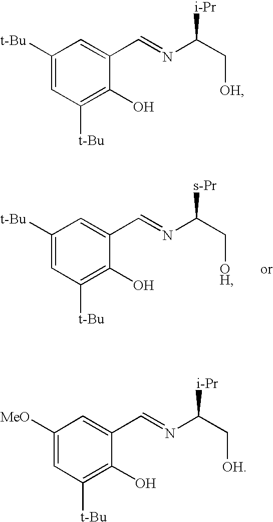

7. The method according to claim 4, wherein the optically active ligand is a reduced Schiff base ligand of formula (III).

8. The method according to claim 7, wherein the reduced Schiff base ligand is selected from the group consisting of

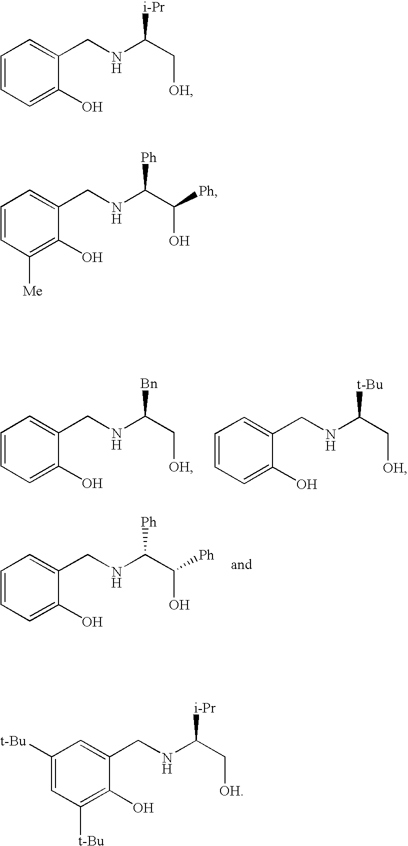

9. The method according to claim 1, wherein the titanium tetraalkoxide compound is represented by the general formula (IV)
[Ti(OR^{a})₄] (IV)
wherein R^{a} is an optionally substituted alkyl group or an optionally substituted aryl group.

10. (canceled)

11. The method according to claim 1, wherein the water source is selected from the group consisting of H₂O, Na₂B₄O₇.10H₂O, Na₂SO₄.10H₂O, MgSO₄.7H₂O, Na₃PO₄.12H₂O, CuSO₄.5 H₂O, FeSO₄.7H₂O AlNa(SO₄)₂.12H₂O, AlK(SO₄)₂.12H₂O and moisture absorbed molecular sieves.

12. The method according to claim 1, wherein the aldehyde or the unsymmetrical ketone is represented by the general formula (V) 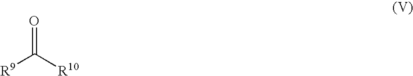
wherein R⁹ and R¹⁰ are different groups, and each represents a hydrogen atom, an alkyl group, an alkenyl group, an alkynyl group, an aryl group, an aromatic heterocyclic group or a non-aromatic heterocyclic group, each of which may be optionally substituted; and R⁹ and R¹⁰ may be linked together to form a ring.

13. The method according to claim 12, wherein the aldehyde is selected from the group consisting of aliphatic aldehydes, α,β-unsaturated aldehydes and aromatic aldehydes.

14. The method according to claim 13, wherein the aldehyde is selected from the group consisting of propionaldehyde, butylaldehyde, valeraldehyde, isovaleraldehyde, hexaaldehyde, heptaldehyde, octylaldehyde, nonylaldehyde, decylaldehyde, isobutylaldehyde, 2-methylbutylaldehyde, 2-ethylbutylaldehyde, 2-ethylhexanal, pivalaldehyde, 2,2-dimethylpentanal, cyclopropanecarboaldehyde, cyclohexanecarboaldehyde, phenylacetaldehyde, (4-methoxyphenyl)acetaldehyde, 3-phenylpropionaldehyde, benzyloxyacetaldehyde, crotonaldehyde, 3-methylcrotonaldehyde, methacrolein, trans-2-hexenal, trans-cinnamaldehyde, benzaldehyde, o-, m- or p-tolylaldehyde, 2,4,6-trimethylbenzaldehyde, 4-biphenylcarboaldehyde, o-, m- or p-fluorobenzaldehyde, o-, m- or p-chlorobenzaldehyde, o-, m- or p-bromobenzaldehyde, 2,3-, 2,4- or 3,4-dichlorobenzaldehyde, 4-(trifluoromethyl)benzaldehyde, 3- or 4-hydroxybenzaldehyde, 3,4-dihydroxybenzaldehyde, o-, m- or p-anisaldehyde, 3,4-dimethoxybenzaldehyde, 3,4-(methylenedioxy)benzaldehyde, m- or p-phenoxybenzaldehyde, m- or p-benzyloxybenzaldehyde, 2,2-dimethylchromane-6-carboaldehyde, 1- or 2-naphthaldehyde, 2- or 3-furancarboaldehyde, 2- or 3-thiophenecarboaldehyde, 1-benzothiophene-3-carboaldehyde, N-methylpyrrole-2-carboaldehyde, 1-methylindole-3-carboaldehyde, 2-, 3- and 4-pyridinecarboaldehyde.

15. The method according to claim 12, wherein the asymmetrical ketone is selected from the group consisting of 2-butanone, 2-pentanone, 2-hexanone, 2-heptanone, 2-octanone, isopropylmethyl ketone, cyclopentylmethyl ketone, cyclohexylmethyl ketone, phenylacetone, p-methoxyphenylacetone, 4-phenylbutane-2-on, cyclohexylbenzyl ketone, acetophenone, o-, m- or p-methylacetophenone, 4-acetylbiphenyl, o-, m- or p-fluoroacetophenone, o-, m- or p-chloroacetophenone, o-, m- or p-bromoacetophenone, 2′,3′-, 2′,4′- or 3′,4′-dichloroacetophenone, m- or p-hydroxyacetophenone, 3′,4′-dihydroxyacetophenone, o-, m- or p-methoxyacetophenone, 3′,4′-dimethoxyacetophenone, m- or p-phenoxyacetophenone, 3′,4′-diphenoxyacetophenone, m- or p-benzyloxyacetophenone, 3′,4′-dibenzyloxyacetophenone, 2-chloroacetophenone, 2-bromoacetophenone, propiophenone, 2-methylpropiophenone, 3-chloropropiophenone, butyrophenone, phenylcyclopropyl ketone, phenylcyclobutyl ketone, phenylcyclopentyl ketone, phenylcyclohexyl ketone, 1- or 2-acenaphthone, chalcone, 1-indanone, 1- or 2-tetralon, 4-chromanone, trans-4-phenyl-3-butene-2-on, 2- or 3-acetylfuran, 2- or 3-acetylthiophene, 2-, 3- and 4-acetylpyridine.

16. The method according to claim 1, wherein the cyanating agent is selected from the group consisting of hydrogen cyanide, acetone cyanohydrin, cyanoformate esters, acetyl cyanide, dialkylcyanophosphates, trialkylsilyl cyanide and benzoyl cyanide.

17. The method according to claim 16, wherein the cyanating agent is methyl cyanoformate or ethyl cyanoformate.

18. The method according to claim 16, wherein the cyanating agent is used in an amount selected from the group consisting of an amount of about 1 to about 3 mol with respect to the amount of the aldehyde or asymmetrical ketone, an amount of about 1.5 to about 2.5 mol with respect to the amount of the aldehyde or asymmetrical ketone.

19. (canceled)

20. The method according to claim 1, wherein the Lewis base is selected from the group consisting of NR¹¹R¹²R¹³, O═NR¹¹R¹²R¹³, dialkylaminopyridine, diarylaminopyridine and N,N,N,N-tetramethylethylenediamine, wherein R¹¹, R¹² and R¹³ are independently selected from the group consisting of hydrogen, alkyl and aryl.

21. The method according to claim 20, wherein the Lewis base is triethylamine or 4-dimethylaminopyridine.

22. The method according to claim 20, wherein the amount of Lewis base used with respect to the amount of the aldehyde or asymmetrical ketone is selected from the group consisting of about 1 to about 10 mol % of the Lewis base with respect to the amount of the aldehyde or asymmetrical ketone, about 1 to about 5 mol % of the Lewis base with respect to the amount of the aldehyde or asymmetrical ketone, and about 1 to about 3 mol % of the Lewis base with respect to the amount of the aldehyde or asymmetrical ketone.

23. (canceled)

24. (canceled)

25. The method according to claim 1, wherein the reaction is carried out in a temperature range selected from the group consisting of a temperature range from about −10° C. to about 40° C., from about 15° C. to about 30° C., and from about 20° C. to about 25° C.

26. (canceled)

27. (canceled)

28. (canceled)

29. (canceled)

30. (canceled)

31. (canceled)

32. The method according to claim 1, wherein the mole fraction of titanium to the optically active ligand is selected from the group consisting of about 0.5≦Ti/ligand≦about 4, about 1≦Ti/ligand≦about 3, and about Ti/ligand=2.

33. (canceled)

34. (canceled)

35. The method according to claim 1, wherein about 1 to about 10 mol % of the optically active catalyst or about 3 to about 5 mol % of the optically active catalyst in terms of the titanium atom with respect to the amount of the aldehyde or asymmetrical ketone is used.

36. (canceled)
